SEQUENCE LISTING (15 sequences)

SEQ ID NO: 1 - DNA, 18 bp, synthetic construct
  source 1..18: mol_type = other DNA
actcctacgggaggcagc

SEQ ID NO: 2 - DNA, 18 bp, synthetic construct
  source 1..18: mol_type = other DNA
gctgcctcccgtaggagt

SEQ ID NO: 3 - DNA, 18 bp, synthetic construct
  source 1..18: mol_type = other DNA
gcagccacccgtaggtgt

SEQ ID NO: 4 - DNA, 18 bp, synthetic construct
  source 1..18: mol_type = other DNA
gctgccacccgtaggtgt

SEQ ID NO: 5 - DNA, 18 bp, synthetic construct
  source 1..18: mol_type = other DNA
catccggcattaccaccc

SEQ ID NO: 6 - DNA, 20 bp, synthetic construct
  source 1..20: mol_type = other DNA
ggtattagcayctgtttcaa

SEQ ID NO: 7 - DNA, 17 bp, synthetic construct
  source 1..17: mol_type = other DNA
ccaatgtgggggacctt

SEQ ID NO: 8 - DNA, 19 bp, synthetic construct
  source 1..19: mol_type = other DNA
gcttcttagtcargtaccg

SEQ ID NO: 9 - DNA, 18 bp, synthetic construct
  source 1..18: mol_type = other DNA
tcagacttgccgyaccgc

SEQ ID NO: 10 - DNA, 17 bp, synthetic construct
  source 1..17: mol_type = other DNA
ggtcggtctctcaaccc

SEQ ID NO: 11 - DNA, 19 bp, synthetic construct
  source 1..19: mol_type = other DNA
attgcgttaactccggcac

SEQ ID NO: 12 - DNA, 18 bp, synthetic construct
  source 1..18: mol_type = other DNA
cgcctacctctgcactac

SEQ ID NO: 13 - DNA, 16 bp, synthetic construct
  source 1..16: mol_type = other DNA
tacggatttcactcct

SEQ ID NO: 14 - DNA, 23 bp, synthetic construct
  source 1..23: mol_type = other DNA
ttatgcggtattaatctyccttt

SEQ ID NO: 15 - DNA, 19 bp, synthetic construct
  source 1..19: mol_type = other DNA
gttagccgtccctttctgg

(19) United States
(12) Patent Application Publication
(10) Pub. No.: US 2025/0205252 A1
(43) Pub. Date: Jun. 26, 2025

(54) 7-KETOLITHOCHOLIC ACID FOR USE IN THE TREATMENT OF GUT DYSBIOSIS AND AS A PREBIOTIC

(71) Applicant: NZP UK Limited, Bristol (GB)

(72) Inventors: Alexander Charles WEYMOUTH-WILSON, Shinfield (GB); Kimberly Ann WATSON, Reading (GB); Glenn GIBSON, Reading (GB); Abigail Rose BASSON, Reading (GB)

(21) Appl. No.: 18/851,912

(22) PCT Filed: Mar. 31, 2023
(86) PCT No.: PCT/GB2023/050846

§ 371 Date: Sep. 27, 2024

(30) Foreign Application Priority Data
Mar. 31, 2022 (GB) 2204688.2
Jan. 18, 2023 (GB) 2300750.3

(51) Int. Cl. A61K 31/575; A61K 45/06; A61K 47/54; A61P 3/08

(52) U.S. Cl. CPC A61K 31/575; A61K 45/06; A61K 47/542; A61P 3/08

(57) ABSTRACT

7-ketolithocholic acid (7-KCLA, 3α-hydroxy-7-oxo-5β-cholanic acid) and its pharmaceutically acceptable salts and glycine or taurine conjugates are useful for the prevention or treatment of gut dysbiosis, diseases and conditions associated with poor gut health, or in improving the health of the gut microbiome.

The present invention relates to a compound which exhibits a prebiotic effect. In particular, the invention relates to 7-ketolithocholic acid and its salts and glycine and taurine conjugates for use as a prebiotic, in particular for improving the health of the gut microbiome and for treating or gut dysbiosis and diseases and conditions associated with poor gut health. The invention also relates to prebiotic compositions comprising 7-ketolithocholic acid and salts and glycine and taurine conjugates thereof.

### BACKGROUND OF THE INVENTION

The gut microbiome consists of the microbial community found in the small and large intestines. The microorganisms in the gut are collectively known as the microbiota and play a vital role in human and animal health and, indeed, can affect the development and course of a number of diseases and conditions (Vyas, 2012). The number of species in the gut microbiota varies from individual to individual but over 1000 different species of gut bacteria have been identified, while there are typically around 160 species in the gut of an individual (Rajilić-Stojanović, 2014). Examples of species which may be found in the gut microbiome include:
beneficial species such as Bifidobacterium species, Lactobacillus species, Roseburia species, Eubacterium rectale and Faecalibacterium prausnitzii;
commensal species such as Bacteroides and Enterococcus; and
pathogenic species such as Desulfovibrionales, Clostridium histolyticum and Atopobium and Coriobacterium species.

A healthy gut microbiome is considered by Wilmanski (2021) to be one that successfully maintains long-term stability, resists invasive pathogens, supplies key nutrients (including vitamins and fermentation byproducts) to its host, and helps maintain host metabolic and immunological homeostasis. Indicators of a healthy gut microbiome include high levels of certain beneficial species of microorganisms, low levels of detrimental microbial species and a diversity of microorganism species.

The gut microbiota is essential for host digestion and complements the activity of mammalian enzymes in the liver and gut mucosa (Rowland, 2018). For example, it plays a role in the breakdown of polysaccharides and polyphenols and the synthesis of vitamins. There is also evidence to suggest that the gut microbiota plays a role in a range of gastrointestinal diseases and conditions such as inflammatory bowel disease (IBD), irritable bowel syndrome (IBS), colon cancer, and antibiotic-associated diarrhoea as well as in obesity and diabetes (Greenblum, 2012; Marchesi, 2016).

Bacterial fermentation of dietary carbohydrates and resistant starches in the large intestine produces short chain fatty acids, especially lactate, acetate, propionate and butyrate.

Butyrate is produced by Faecalibacterium spp., Roseburia spp. and Eubacterium spp. It is used in the Acetyl COA-Butyl CoA pathway and both increases satiety and decreases inflammation. Other beneficial effects of butyrate include prevention and treatment of diet-induced obesity and insulin resistance (Steliou, 2012; De Vadder, 2014), which has implications for the prevention of metabolic syndrome and type II diabetes mellitus. Butyrate can also enhance the gut barrier function of intestinal epithelial cells, exert anti-inflammatory effects (Ma, 2012; Matter, 2005) and, furthermore, it has potential anti-cancer activity (Steliou, 2012; Gonçalves, 2013).

Gut dysbiosis refers to altered bacterial colonisation of the gut associated with disease expression. The altered composition may comprise a decrease in the number of beneficial microorganisms, for example beneficial bacteria, an increase in the number of pathogenic microorganisms, for example pathogenic bacteria, or a decrease in the overall number of microorganisms making up the gut microbiota. Studies have shown that gut dysbiosis can lead to one or more conditions selected from:
obesity, hyperglycemia, metabolic syndrome, pre-diabetes and type 2 diabetes mellitus (Belizário, 2018; Wu, 2020), inflammatory bowel diseases, including ulcerative colitis and Crohn's disease, irritable bowel syndrome, and other inflammatory conditions as well as cardiovascular and central nervous system disorders, including anxiety and depression (Belizário, 2018, Flux, 2021), bipolar disorder (Wilmanski, 2021), sleep disorders and cognitive dysfunction (Pferschy-Wenzig, 2022) and schizophrenia (Flux, 2021). Martinez, (2021) teaches that gut dysbiosis is believed to contribute to the development of various immune-mediated conditions, including inflammatory bowel disease (IBD), rheumatoid arthritis, type 1 diabetes mellitus, multiple sclerosis, coeliac disease and systemic lupus erythematosus (SLE).

Mahmud (2022) teaches that there is a link between the gut microbiota and the skin microbiota and that gut dysbiosis can contribute to skin conditions including psoriasis, atopic dermatitis, acne vulgaris, rosacea, alopecia areata and hidradenitis suppurativa.

There is also evidence that gut dysbiosis can lead to cancer, for example cancer of the colon (Belizário, 2018) and liver disease.

Probiotics are defined as “live microorganisms that, when administered in adequate amounts, confer a health benefit on the host” (Hill, 2014) and probiotic microorganisms include Bifidobacterium and Lactobacillus strains as well as Akkermansia muciniphila, Faecalibacterium prausnitzii and Roseburia spp.

Work has also been carried out on prebiotics, which were originally defined as nondigestible food ingredients that beneficially affect the host by selectively stimulating the growth and/or activity of one or a limited number of bacterial species already resident in the colon, and thus attempt to improve host health (Gibson, 1995). The first prebiotics were almost exclusively dietary fibres but, more recently, it has been appreciated that other substances may have prebiotic effects and prebiotics have recently been redefined as a substance which can be selectively utilised by host microorganisms to confer a health benefit (Gibson, 2017).

Bile acids play a role in the metabolism of glucose and lipids in humans and other mammals via a complex signalling system. The primary bile acids, cholic acid (CA) and chenodeoxycholic acid (CDCA), are synthesised in the liver from cholesterol. Prior to secretion into bile, taurine or glycine conjugates (primary bile salts) of the primary bile acids are formed and these are then transported into the gall bladder prior to secretion into the stomach as part of the digestive process. The majority of bile acids are recycled back to the liver in the enterohepatic circulation, but a small proportion enters the colon and interacts with the gut microbiota.

Primary bile acids and bile salts are essential for the absorption of fat and fat-soluble vitamins as part of the digestion process and they act as metabolic sensing molecules, signalling through G protein coupled receptors (GPCRs) and nuclear receptors (NRs) to regulate the homeostasis of lipids and glucose. Primary bile acids and bile salts have detergent properties that have an anti-bacterial action which suppresses microbial blooms in the gut. CA and CDCA are converted to secondary bile acids deoxycholic acid (DCA) and lithocholic acid (LCA) by gut bacteria and these secondary bile acids act as a second series signalling molecules.

It has recently been discovered that, in addition to their role in metabolism of glucose and lipids, bile acids also affect the gut microbiome. For example, male patients who previously had colorectal adenomas removed and subsequently treated with ursodeoxycholic acid (UDCA) over a period of three years were found to show an increase in the incidence of the beneficial microorganism Faecalibacterium prausnitzii, (Pearson, 2019). UDCA also has been shown to reduce the levels of hydrogen- and methane-producing bacteria in the small intestine (Kim, 2020). It has therefore been suggested that there is potential to use UDCA as a prebiotic substance that can be selectively utilised by host microorganisms to promote beneficial bacteria in the gut and lead to a health benefit.

The bile acid 7-ketolithocholic acid (7-KLCA) is metabolised in the gut to produce UDCA and the inventors have surprisingly discovered that 7-KLCA has particularly favourable prebiotic properties, conferring a positive effect on the growth of beneficial micororganisms of the gut microbiota.

### SUMMARY OF THE INVENTION

In a first aspect of the invention there is provided 7-ketolithocholic acid (7-KCLA, 3α-hydroxy-7-oxo-5β-cholanic acid) having the formula:

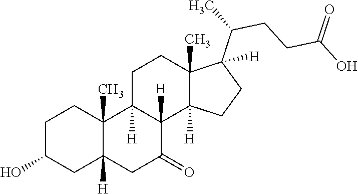

or a salt, especially a pharmaceutically acceptable salt, or a glycine or taurine conjugate thereof for use in the prevention or treatment of gut dysbiosis and diseases and conditions associated with poor gut health, or for use in improving the health of the gut microbiome.

Surprisingly, it has been found that 7-KLCA is able to improve the health of the gut microbiome by selectively promoting the growth of beneficial microorganisms, especially beneficial bacterial species.

Therefore, there is also provided a method of adjusting the composition of the gut microbiota of a subject to comprise a greater proportion of beneficial microorganisms, the method comprising administering to the subject 7-KLCA or a salt, especially a pharmaceutically acceptable salt, or a glycine or taurine conjugate thereof.

### Definitions

In the present specification, except where the context requires otherwise due to express language or necessary implication, the word “comprises”, or variations such as “comprises” or “comprising” is used in an inclusive sense i.e. to specify the presence of the stated features but not to preclude the presence or addition of further features in various embodiments of the invention.

In the present specification, a prebiotic is a compound that promotes the growth of beneficial 5 microorganisms in the intestines, exhibiting a prebiotic effect. Beneficial microorganisms are discussed in greater detail below and comprise, in particular, beneficial bacteria, including, but not limited to Bifidobacteria species, Faecalibacterium prausnitzii, Eubacterium rectale, Roseburia species, Lactobacillus species, Bacteroides xylanisolvens and Eubacterium eligens.

The subject to which the prebiotic is administered is most suitably a human but, in some cases, it may be used in treating other animals, particularly for agricultural purposes. For example, it may be used to treat mammals including pigs and ruminants such as cows, sheep and goats; birds, for example chickens, turkeys and geese; and aquatic animals such as fish.

In the present specification, “improving the health of the gut microbiome” refers to adjusting the composition of the gut microbiota of a subject to comprise a greater proportion of beneficial microorganisms, especially beneficial bacteria such as those mentioned above.

In some cases, improving the health of the gut microbiome may also include adjusting the composition of the gut microbiota of a subject to comprise a smaller proportion of detrimental microorganisms, especially detrimental bacteria. Detrimental bacteria include Atopobium, Coriobacterium, Clostridium histolyticum, Clostridium perfringens, Desulfovibrionales, Desulfuromonadales and Dialister pneumosintes, especially Atopobium, Coriobacterium, Clostridium histolyticum, Clostridium perfringens, Desulfovibrionales and Desulfuromonadales.

Improving the health of the gut microbiome may also include increasing the total number of microorganisms in the gut microbiota.

In the present specification, a patient with pre-diabetes has a fasting plasma glucose level of 100 to 125 mg/dl and a patient with diabetes has a fasting plasma glucose level of 126 mg/dl or higher (American Diabetes Association).

According to the International Diabetes Federation, a patient with metabolic syndrome has a combination of central obesity and any two of the following high blood pressure (130/85 mm Hg or higher), high levels of plasma triglyceride (≥150 mg/dL) and low levels of plasma HDL cholesterol (<40 mg/dL) and raised fasting plasma glucose (>5.6 mmol/L or 100 mg/dL).

“Hyperglycaemia” refers to a plasma glucose level of 11.1 mmol/L (200 mg/dL).

Salts of 7-KLCA are suitably non-toxic and pharmaceutically acceptable. Suitable pharmaceutically acceptable salts are well known to those of skill in the art and are described, for example by Gupta et al. (2018). Some particularly suitable salts of the compounds of general formula (I) include basic addition salts such as sodium, potassium, calcium, aluminium, zinc, magnesium and other metal salts as well as choline, diethanolamine, ethanolamine, ethyl diamine and meglumine salts.

In taurine conjugates of bile acids, including 7-KLCA, the side chain carboxylic acid group is replaced by: C(O)NHCH₂CH₂S(O)₂OH, while in glycine conjugates the carboxylic acid group is replaced by C(O)NHCH₂C(O)OH. In vivo, Bile acids are often found as their glycine or taurine conjugates and conjugated bile acids are the major components of human bile.

A person of skill in the art would therefore been aware that a salt or a glycine or taurine conjugate of 7-KLCA would be expected to have substantially the same activity in vivo as the parent bile acid.

### DETAILED DESCRIPTION OF THE INVENTION

The invention provides 7-KLCA or a salt, especially a pharmaceutically acceptable salt, or a glycine or taurine conjugate thereof for use in the prevention or treatment of gut dysbiosis and diseases and conditions associated with poor gut health, or for use in improving the health of the gut microbiome.

In one aspect, there is provided 7-KLCA or a salt, especially a pharmaceutically acceptable salt, or a glycine or taurine conjugate thereof for use in the prevention of gut dysbiosis.

In a further aspect there is provided 7-KLCA or a salt, especially a pharmaceutically acceptable salt, or a glycine or taurine conjugate thereof for use in the treatment of gut dysbiosis.

There is also provided in another aspect 7-KLCA or a salt, especially a pharmaceutically acceptable salt, or a glycine or taurine conjugate thereof for use in the prevention of diseases and conditions associated with poor gut health.

In still another aspect, there is provided 7-KLCA or a salt, especially a pharmaceutically acceptable salt, or a glycine or taurine conjugate thereof for use in the treatment of diseases and conditions associated with poor gut health.

In a further aspect, there is provided 7-KLCA or a salt, especially a pharmaceutically acceptable salt, or a glycine or taurine conjugate thereof for use in improving the health of the gut microbiome.

There is also provided the use of 7-KLCA or a pharmaceutically acceptable salt or a glycine or taurine conjugate thereof in the manufacture of a medicament for the prevention or treatment of gut dysbiosis and diseases and conditions associated with poor gut health, or for use in improving the health of the gut microbiome.

In an aspect of the invention, there is provided the use of 7-KLCA or a salt, especially a pharmaceutically acceptable salt, or a glycine or taurine conjugate thereof in the manufacture of a medicament for the prevention of gut dysbiosis.

In a further aspect, the invention provides the use of 7-KLCA o or a salt, especially a pharmaceutically acceptable salt, or a glycine or taurine conjugate thereof in the manufacture of a medicament for the treatment of gut dysbiosis.

In another aspect, there is provided the use of 7-KLCA or a salt, especially a pharmaceutically acceptable salt, or a glycine or taurine conjugate thereof in the manufacture of a medicament for the prevention of diseases and conditions associated with poor gut health.

There is also provided the use of 7-KLCA or a salt, especially a pharmaceutically acceptable salt, or a glycine or taurine conjugate thereof in the manufacture of a medicament for treatment of diseases and conditions associated with poor gut health.

In still a further aspect, the invention provides the use of 7-KLCA or a salt, especially a pharmaceutically acceptable salt, or a glycine or taurine conjugate thereof in the manufacture of a medicament for use in improving the health of the gut microbiome.

There is further provided a method for the prevention or treatment of gut dysbiosis and diseases and conditions associated with poor gut health, or for improving the health of the gut microbiome, the method comprising administering to a subject an effective amount of 7-KLCA or a salt, especially a pharmaceutically acceptable salt, or a glycine or taurine conjugate thereof.

In an aspect of the invention, there is provided a method for the prevention of gut dysbiosis, the method comprising administering to a subject an effective amount of 7-KLCA or a salt, especially a pharmaceutically acceptable salt, or a glycine or taurine conjugate thereof.

In a further aspect there is provided a method for the treatment of gut dysbiosis, the method comprising administering to a subject an effective amount of 7-KLCA or a salt, especially a pharmaceutically acceptable salt, or a glycine or taurine conjugate thereof.

In still another aspect, there is provided a method for the prevention of diseases and conditions associated with poor gut health, the method comprising administering to a subject an effective amount of 7-KLCA or a salt, especially a pharmaceutically acceptable salt, or a glycine or taurine conjugate thereof.

In another aspect, there is provided a method for the treatment of diseases and conditions associated with poor gut health, the method comprising administering to a subject an effective amount of 7-KLCA or a salt, especially a pharmaceutically acceptable salt, or a glycine or taurine conjugate thereof.

In yet a further aspect, there is provided a method for improving the health of the gut microbiome, the method comprising administering to a subject an effective amount of 7-KLCA or a salt, especially a pharmaceutically acceptable salt, or a glycine or taurine conjugate thereof.

As noted above, gut dysbiosis is associated with a number of diseases and conditions such as obesity, hyperglycemia, insulin resistance, metabolic syndrome, pre-diabetes, type 2 diabetes, inflammatory bowel diseases including ulcerative colitis and Crohn's disease, irritable bowel syndrome and autoimmune conditions including rheumatoid arthritis, multiple sclerosis, type I diabetes, coeliac disease, systemic lupus erythematosus, CNS disorders including anxiety, depression, sleep disorders cognitive dysfunction, schizophrenia and bipolar disorder, cardiovascular disease and skin conditions including psoriasis, atopic dermatitis, acne vulgaris, rosacea, alopecia areata and hidradenitis suppurativa.

In some cases, gut dysbiosis may arise from the treatment of bacterial infections with an antibiotic. When an antibiotic is administered to treat a bacterial infection, it can have negative effects on the gut microbiota, including reduced species diversity and altered metabolic activity, often resulting from antibiotic-associated diarrhoea, as well as a reduction in the overall number of microorganisms present. It can also lead to the proliferation of antibiotic-resistant organisms in the gut microbiota, which may be particularly relevant for long-term or frequent antibiotic use.

Because 7-KLCA and its salts and glycine and taurine conjugates promote the proliferation of beneficial microorganisms, for example bacterial species such as Bifidobacteria species, Faecalibacterium prausnitzii, Eubacterium rectale, Roseburia species, Lactobacillus species, Bacteroides xylanisolvens and Eubacterium eligens, especially Bifidobacteria species, Faecalibacterium prausnitzii and Roseburia species, while inhibiting the proliferation of harmful species such as Atopobium, Coriobacterium, Clostridium histolyticum, Clostridium perfringens, Desulfovibrionales, Desulfuromonadales and Dialister pneumosintes, especially Atopobium, Coriobacterium, Clostridium histolyticum, Clostridium perfringens, Desulfovibrionales and Desulfuromonadales 7-KLCA and its salts, especially its pharmaceutically acceptable salts, and glycine and taurine conjugates are expected to be useful in restoring the health of the gut microbiome after treatment with an antibiotic.

Therefore, the 7-KLCA may be administered in combination with or after the administration of an antibiotic in order to ensure that the gut microbiome is restored to a healthy state after a bacterial infection.

7-KLCA and its salts and glycine or taurine conjugates are effective to increase the number of beneficial microorganisms and reduce the number of detrimental microorganisms in the gut and to prevent or treat a number of diseases and conditions associated with poor gut health. The inventors have shown that 7-KLCA is able to selectively enhance Bifidobacteria species, Faecalibacterium prausnitzii, Eubacterium rectale, Roseburia species, Lactobacillus species, Bacteroides xylanisolvens and Eubacterium eligens, especially Bifidobacteria species, Faecalibacterium prausnitzii and Roseburia species in the gut microbiome (see Examples 1 and 2 below). This is particularly advantageous and, as shown in Example 2 and contrary to the findings of Pearson 2019, is not found following administration of other bile acids, such as UDCA, at least over the timescale of the inventors' investigation. The inventors have also shown that 7-KLCA reduces the incidence of pathogenic microorganisms in the gut, for example bacterial species including Atopobium, Coriobacterium, Clostridium histolyticum, Clostridium perfringens, Desulfovibrionales and Desulfuromonadales (again, see Example 2). Bifidobacteria, for example Bifidobacterium longum subsp. longum are known to have beneficial effects (Wong, 2019) on the gut microbiome, and have been incorporated into various probiotic products.

Faecalibacterium prausnitzii is also known to be beneficial, particularly in reducing metabolic disorders including obesity, hyperglycemia, insulin resistance, pre-diabetes, type 2 diabetes and gut inflammation. It has been found that F. prausnitzii is able to improve gut homeostasis and influence insulin sensitivity through production of the SCFA (Short Chain Fatty Acid) butyrate (Maioli, 2021; Wu, 2020). A reduction in SCFA-producing species such as Faecalibacterium prausnitzii is also linked to bipolar disorder (Wilmanski, 2021). The gut microbiome is associated with various neurocognitive and mental health conditions via a microbiome-gut-brain axis (Kraeuter, 2020; Flux, 2021; Pferschy-Wenzig, 2022). Improving the health of the gut microbiome may therefore prevent or ameliorate neurocognitive and mental health related conditions including anxiety, depression, sleep disorders cognitive dysfunction, schizophrenia and related psychotic disorders.

Roseburia species are known to improve colonic motility, immune function, and to have anti-inflammatory properties. It has been suggested that Roseburia species affect various metabolic pathways and that they are of use in preventing and treating irritable bowel syndrome, obesity, pre-diabetes, type 2 diabetes and allergies (Tamanai-Shacoori, 2017).

Bifidobacteria produces the SCFA acetate which, in turn acts as a “fuel” to butyrate producing species such as Faecalibacterium prausnitzii and Roseburia species. Prediabetic patients have an altered gut microbiome with reduced levels of butyrate producing bacteria and this altered microbiome plays a role in disease progression to type 2 diabetes. Also, as noted above, butyrate can also enhance the gut barrier function of intestinal epithelial cells, exert anti-inflammatory effects (Ma, 2012; Matter, 2005) and, furthermore, it has potential anti-cancer activity (Steliou, 2012; Gonçalves, 2013).

There also appears to be a link between the gut microbiota and the skin microbiota (Mahmud, 2022) such that disruption of gut integrity, and an imbalance within microbial communities can have a significant impact on the overall homeostasis of skin, which means that improving the health of the gut microbiome may be beneficial for the treatment or prevention of skin diseases. For example, Faecalibacterium prausnitzii and Ruminococcus species have been found to be protective against psoriasis via competitive inhibition of pathogenic organisms and production of SCFAs. Lactobacillus species decrease skin inflammation, reduce the size of acne lesions via modulation of the immune system, while both Lactobacillus species and Bifidobacterium species are useful in reducing the severity of atopic dermatitis (eczema). Reduction in the amount of pathogenic species such as Helicobacter pylori reduces rosacea, while the activation of the immune system associated with a healthy gut microbiome leads to the reduction or prevention of alopecia areata and hideradenitis suppurativa (Mahmud, 2022).

A reduction in the species diversity of the gut microbiota is also associated with IBD, obesity and type 2 diabetes (Wilmanski, 2021; (Flux, 2021).

Because of their effect on the gut microbiota, 7-KLCA, its glycine and taurine conjugates and its salts, especially its pharmaceutically acceptable salts, are useful in the prevention and treatment of a number of diseases and conditions associated with poor gut health. These diseases and conditions include obesity, hyperglycemia, insulin resistance, metabolic syndrome, pre-diabetes, type 2 diabetes, inflammatory bowel diseases including ulcerative colitis and Crohn's disease, irritable bowel syndrome and autoimmune conditions including rheumatoid arthritis, multiple sclerosis, type I diabetes, coeliac disease, systemic lupus erythematosus, CNS disorders including anxiety, depression, sleep disorders cognitive dysfunction, schizophrenia and bipolar disorder, cardiovascular disease and skin conditions including psoriasis, atopic dermatitis, acne vulgaris, rosacea, alopecia areata and hidradenitis suppurativa.

In particular, the diseases and conditions include obesity, hyperglycemia, metabolic syndrome, type 2 diabetes, inflammatory bowel diseases including ulcerative colitis and Crohn's disease, irritable bowel syndrome and autoimmune conditions including rheumatoid arthritis, multiple sclerosis, type I diabetes and coeliac disease. More particularly, the diseases and conditions include obesity, hyperglycemia, metabolic syndrome and type 2 diabetes.

WO 2017/019524 and WO 2015/183794 both teach that several bile acids have TGR5 agonist and that, because of this, they are useful for treating diseases and conditions such as obesity, diabetes and metabolic syndrome. There are numerous references in the prior art to the TGR5 agonist activity of bile acids such as taurolithochoic acid, lithocholic acid, deoxycholic acid, chenodeoxycholic acid and cholic acid (e.g. Guo, 2016). WO 2017/019524 also teaches that some bile acids have FXR agonist activity and that FXR may be involved in glucose and insulin metabolism. Bile acids such as chenodeoxycholic acid and bile acid derivatives such as obeticholic acid are known to have FXR agonist activity (Guo, 2018). There is no evidence in the literature, however, that 7-KLCA has significant activity either as a TGR5 agonist or as an FXR agonist and it would not therefore be expected from the teaching of documents such as WO 2017/019524 and WO 2015/183794 that 7-KLCA would be of use in the treatment of TGR5- and FXR-mediated conditions such as obesity, diabetes and metabolic syndrome.

Because of its ability to promote the growth of bifidobacteria and butyrate-producing bacterial species, 7-KLCA and its salts, especially its pharmaceutically acceptable salts, and its glycine and taurine conjugates are also of use in enhancing gut barrier function of intestinal epithelial cells, and as an anti-inflammatory agent.

In a further aspect of the invention there is provided a method of adjusting the composition of the gut microbiota of a subject to comprise a greater proportion of beneficial microorganisms, the method comprising administering to the subject 7-KLCA or a salt, especially a pharmaceutically acceptable salt, or a glycine or taurine conjugate thereof.

The beneficial microorganisms may comprise bacteria, for example, bacteria selected from Bifidobacteria species, Faecalibacterium prausnitzii, Eubacterium rectale, Roseburia species, Lactobacillus species, Bacteroides xylanisolvens and Eubacterium eligens, especially Bifidobacteria species, Faecalibacterium prausnitzii and Roseburia species.

Furthermore, the method may also comprise adjusting the composition of the gut microbiota of the subject to comprise a lower proportion of pathogenic microorganisms. The pathogenic micororganisms may comprise bacteria, for example, bacteria selected from Atopobium, Coriobacterium, Clostridium histolyticum, Clostridium perfringens, Desulfovibrionales Desulfuromonadales and Dialister pneumosintes, especially Atopobium, Coriobacterium, Clostridium histolyticum, Clostridium perfringens, Desulfovibrionales and Desulfuromonadales.

The method may also comprise increasing the number of microorganisms, particularly bacteria, in the gut microbiota.

In some cases, the gut microbiota may require adjustment after the subject has been treated with an antibiotic since, as noted above, antibiotic treatment can have negative effects on the gut microbiota.

The method therefore may comprise administering the 7-KLCA either simultaneously with or after antibiotic treatment.

The antibiotic and the 7-KLCA or a salt, especially a pharmaceutically acceptable salt, or glycine or taurine conjugate thereof may be administered together and may be provided in a single composition. Alternatively, they may be provided as separate compositions which are adapted to be administered separately or sequentially.

Suitably, an antibiotic is selected from the group consisting of a macrolide (e.g. clarithromycin or erythromycin), a penicillin (e.g. nafcillin, ampicillin or amoxicillin), a cephalosporin (e.g. cefazolin), a carbepenem (e.g. imipenem or aztreonam), another beta-lactam antibiotic, a beta-lactam inhibitor (e.g. sulbactam), an oxaline (e.g. linezolid), an aminoglycoside (e.g. gentamicin), chloramphenicol, a sulfonamide (e.g. sulfamethoxazole), a glycopeptide (e.g. vancomycin), a quinolone (e.g. ciprofloxacin), a tetracycline (e.g. minocycline), fusidic acid, trimethoprim, metronidazole, clindamycin, mupirocin, a rifamycin (e.g. rifampin), a streptogramin (e.g. quinupristin and dalfopristin), a lipoprotein (e.g. daptomycin), a polyene (e.g. amphotericin B), an azole (e.g. fluconazole), and an echinocandin (e.g. caspofungin acetate).

The 7-KLCA or salt, especially a pharmaceutically acceptable salt, or glycine or taurine conjugate thereof is intended for use as a prebiotic and is therefore suitably administered as part of a composition, suitably a composition for oral administration.

Therefore, in a further aspect of the invention there is provided a prebiotic composition comprising 7-KLCA or a salt, especially a pharmaceutically acceptable salt, or glycine or taurine conjugate thereof as the sole active agent together with a suitable carrier.

In some embodiments, the composition does not contain an aqueous soluble starch conversion product obtained by the partial hydrolysis of starch. Examples of aqueous soluble starch conversion products include maltodextrin, dextrin, liquid glucose, corn syrup solid, liquid glucose and soluble starch.

In some embodiments, the composition does not contain maltodextrin.

In some embodiments, the composition does not contain an aqueous soluble non-starch polysaccharide, for example guar gum, pectin, gum arabic, psyllium, oat gum, soybean fibre, oat bran, corn bran, cellulose or wheat bran.

In some embodiments, the composition does not contain guar gum, pectin or gum arabic.

In some embodiments, the composition contains neither an aqueous soluble starch conversion product obtained by the partial hydrolysis of starch nor an aqueous soluble non-starch polysaccharide.

In some embodiments, the composition contains neither maltodextrin nor any one of guar gum, pectin and gum arabic

Suitably, the composition is adapted for oral administration. It may be formulated as discrete units such as capsules, sachets or tablets each containing a predetermined amount of 7-KLCA or a salt, especially a pharmaceutically acceptable salt, or a glycine or taurine conjugate thereof; or as a powder or granules.

For tablets and capsules, suitable carriers include vehicles such as common excipients e.g. binding agents, for example syrup, acacia, gelatin, sorbitol, tragacanth, polyvinylpyrrolidone (Povidone), methylcellulose, ethylcellulose, sodium carboxymethylcellulose, hydroxypropylmethylcellulose, sucrose and starch; fillers and carriers, for example corn starch, gelatin, lactose, sucrose, microcrystalline cellulose, kaolin, mannitol, dicalcium phosphate, sodium chloride and alginic acid; and lubricants such as magnesium stearate, sodium stearate and other metallic stearates, glycerol stearate, stearic acid, silicone fluid, talc waxes, oils and colloidal silica. In some compositions, the excipients are not soluble starch conversion products or soluble non-starch polysaccharides as described above. In particular, in some embodiments, the excipients may not include maltodextrin. In other embodiments, the excipients do not include one or more of guar gum, pectin and gum arabic. Flavouring agents such as peppermint, oil of wintergreen, cherry flavouring and the like can also be used. It may be desirable to add a colouring agent to make the dosage form readily identifiable. Tablets may also be coated by methods well known in the art.

A tablet may be made by compression or moulding, optionally with one or more accessory ingredients. Compressed tablets may be prepared by compressing in a suitable machine the active agent in a free flowing form such as a powder or granules, optionally mixed with a binder, lubricant, inert diluent, preservative, surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active agent.

Other formulations suitable for oral administration include lozenges comprising 7-KLCA or a salt, especially a pharmaceutically acceptable salt, or glycine or taurine conjugate thereof in a flavoured base, usually sucrose and acacia or tragacanth; pastilles comprising 7-KLCA or a salt, especially a pharmaceutically acceptable salt, or glycine or taurine conjugate thereof in an inert base such as gelatin and glycerin, or sucrose and acacia.

Alternatively, the composition may be a food or drink product comprising 7-KLCA or a salt, especially a pharmaceutically acceptable salt, or glycine or taurine conjugate thereof and a suitable flavouring. Food products include, for example yoghurts or breads, while drinks may be milk based or water-based and preferably contain a flavouring and/or taste masking agent to mask the bitter taste of 7-KLCA.

As noted above, one of the uses of 7-KLCA and its salts, especially its pharmaceutically acceptable salts, and glycine and taurine conjugates is to restore the gut microbiome to a healthy state during or after the administration of an antibiotic. Therefore, the composition may further comprise an antibiotic, for example one of the antibiotics listed above.

There is also provided a prebiotic composition comprising 7-KLCA or a salt, especially a pharmaceutically acceptable salt, or glycine or taurine conjugate thereof and a suitable carrier for use in the prevention or treatment of gut dysbiosis, diseases and conditions associated with poor gut health, or for use in improving the health of the gut microbiome.

In one aspect, there is provided a prebiotic composition comprising 7-KLCA or a salt, especially a pharmaceutically acceptable salt, or a glycine or taurine conjugate thereof for use in the prevention of gut dysbiosis.

In a further aspect there is provided a prebiotic composition comprising 7-KLCA or a salt, especially a pharmaceutically acceptable salt, or a glycine or taurine conjugate thereof for use in the treatment of gut dysbiosis.

There is also provided in another aspect a prebiotic composition comprising 7-KLCA or a salt, especially a pharmaceutically acceptable salt, or a glycine or taurine conjugate thereof for use in the prevention of diseases and conditions associated with poor gut health.

In still another aspect, there is provided a prebiotic composition comprising 7-KLCA or a salt, especially a pharmaceutically acceptable salt, or a glycine or taurine conjugate thereof for use in the treatment of diseases and conditions associated with poor gut health.

In a further aspect, there is provided a prebiotic composition comprising 7-KLCA or a salt, especially a pharmaceutically acceptable salt, or a glycine or taurine conjugate thereof for use in improving the health of the gut microbiome.

The invention further provides the use of a prebiotic composition comprising 7-KLCA or a pharmaceutically acceptable salt or glycine or taurine conjugate thereof and a suitable carrier in the manufacture of a medicament for the prevention or treatment of gut dysbiosis, diseases and conditions associated with poor gut health, or for use in improving the health of the gut microbiome.

In an aspect of the invention, there is provided the use of a prebiotic composition comprising 7-KLCA or a salt, especially a pharmaceutically acceptable salt, or a glycine or taurine conjugate thereof in the manufacture of a medicament for the prevention of gut dysbiosis.

In a further aspect, the invention provides the use of a prebiotic composition comprising 7-KLCA or a salt, especially a pharmaceutically acceptable salt, or a glycine or taurine conjugate thereof in the manufacture of a medicament for the treatment of gut dysbiosis.

In another aspect, there is provided the use of a prebiotic composition comprising 7-KLCA or a salt, especially a pharmaceutically acceptable salt, or a glycine or taurine conjugate thereof in the manufacture of a medicament for the prevention of diseases and conditions associated with poor gut health.

There is also provided the use of a prebiotic composition comprising 7-KLCA or a salt, especially a pharmaceutically acceptable salt, or a glycine or taurine conjugate thereof in the manufacture of a medicament for treatment of diseases and conditions associated with poor gut health.

In still a further aspect, the invention provides the use of a prebiotic composition comprising 7-KLCA or a salt, especially a pharmaceutically acceptable salt, or a glycine or taurine conjugate thereof in the manufacture of a medicament for use improving the health of the gut microbiome.

There is also provided a method for the prevention or treatment of gut dysbiosis, diseases and conditions associated with poor gut health, or for use in improving the health of the gut microbiome, the method comprising administering to the subject a prebiotic composition comprising 7-KLCA or a salt or a glycine or taurine conjugate thereof and a suitable carrier.

In an aspect of the invention, there is provided a method for the prevention of gut dysbiosis, the method comprising administering to a subject an effective amount of a prebiotic composition comprising 7-KLCA or a salt, especially a pharmaceutically acceptable salt, or a glycine or taurine conjugate thereof.

In a further aspect there is provided a method for the treatment of gut dysbiosis, the method comprising administering to a subject an effective amount of a prebiotic composition comprising 7-KLCA or a salt, especially a pharmaceutically acceptable salt, or a glycine or taurine conjugate thereof.

In still another aspect, there is provided a method for the prevention of diseases and conditions associated with poor gut health, the method comprising administering to a subject an effective amount of a prebiotic composition comprising 7-KLCA or a salt, especially a pharmaceutically acceptable salt, or a glycine or taurine conjugate thereof.

In another aspect, there is provided a method for the treatment of diseases and conditions associated with poor gut health, the method comprising administering to a subject an effective amount of a prebiotic composition comprising 7-KLCA or a salt, especially a pharmaceutically acceptable salt, or a glycine or taurine conjugate thereof.

In yet a further aspect, there is provided a method for improving the health of the gut microbiome, the method comprising administering to a subject an effective amount of a prebiotic composition comprising 7-KLCA or a salt, especially a pharmaceutically acceptable salt, or a glycine or taurine conjugate thereof.

There is also provided a method of adjusting the composition of the gut microbiota of a subject to comprise a greater proportion of beneficial microorganisms, for example beneficial bacteria, the method comprising administering to the subject a prebiotic composition comprising 7-KLCA or a salt, especially a pharmaceutically acceptable salt, or a glycine or taurine conjugate thereof and a suitable carrier.

As noted above, beneficial bacteria include Bifidobacteria species, Faecalibacterium prausnitzii, Eubacterium rectale, Roseburia species, Lactobacillus species, Bacteroides xylanisolvens and Eubacterium eligens.

The method may further comprise adjusting the composition of the gut microbiota of a subject to comprise a lower proportion of pathogenic microorganisms, especially pathogenic bacteria.

As set out above, pathogenic bacteria include Atopobium, Coriobacterium, Clostridium histolyticum, Clostridium perfringens, Desulfovibrionales, Desulfuromonadales and Dialister pneumosintes.

The method may also comprise increasing the number of microorganisms, particularly bacteria, in the gut microbiota.

Other preferences are as set out above for the medical uses of 7-KLCA or a salt, especially a pharmaceutically acceptable salt, or a glycine or taurine conjugate thereof.

### BRIEF DESCRIPTION OF THE DRAWINGS

FIG. 1 is a plot showing the effects of 7-KLCA and maltodextrin (as the carbon source) on a variety of bacterial species in a batch culture fermentation slurry derived from faecal samples at 0, 5, 10 and 24 hours after inoculation of the slurry. In the figure, EUB refers to total bacterial population; BIF refers to Bifidobacterium species; LAB refers to Lactobacillus—Enterococcus; BAC refers to Bacteroides—Prevotella; EREC refers to Eubacterium rectale—Clostridium coccoides; RREC refers to Roseburia and Eubacterium rectale; ATO refers to Atopobium—Coriobacterium; PROP refers to Clostridial cluster IX; FPRAU refers to Faecalibacterium prausnitzii; DSV refers to Desulfovibrionales and Dessulfuromonadales; and CHIS refers to Clostridium histolyticum—perfringens.

FIG. 2 is a plot showing the effects of maltodextrin (as the carbon source) alone on a variety of bacterial species in a batch culture fermentation slurry derived from faecal samples at 0, 5, 10 and 24 hours after inoculation of the slurry. In the figure, EUB refers to total bacterial population; BIF refers to Bifidobacterium species; LAB refers to Lactobacillus—Enterococcus; BAC refers to Bacteroides—Prevotella; EREC refers to Eubacterium rectale—Clostridium coccoides; RREC refers to Roseburia and Eubacterium rectale; ATO refers to Atopobium—Coriobacterium; PROP refers to Clostridial cluster IX; FPRAU refers to Faecalibacterium prausnitzii; DSV refers to Desulfovibrionales and Dessulfuromonadales; and CHIS refers to Clostridium histolyticum—perfringens.

FIG. 3 is a series of plots showing the production of the SCFAs acetate, propionate, butyrate and lactate in samples of a batch culture fermentation slurry derived from faecal samples at 0 (TO), 5 (T5), 10 (T10) and 24 (T24) hours after inoculation of the slurry with 7-KLCA and maltodextrin together (7-KLCA) or maltodextrin alone (MAL) and an uninoculated control sample (NEG).

FIG. 4 is a series of graphs showing the results of flow-FISH analysis of Example 2 from baseline samples after the first steady state (SS1) was reached after 512 h (8 full volume turnovers, 14 days) and after UDCA and 7KLCA treatment for a further 512 h (8 full volume turnovers, 14 days) from 3-stage continuous culture fermentation systems. Graphs show total bacterial population (EUB-I-II-III), Bifidobacterium spp. (Bif164), Lactobacillus spp. (Lab158) and Bacteroides-Prevotella (Bac303). Graphs show bacterial numbers in log 10 (CFU/mL). Error bars are SEM.

FIG. 5 is a series of graphs showing the results of flow-FISH analysis of Example 2 from baseline samples after the first steady state (SS1) was reached after 512 h (8 full volume turnovers, 14 days) and after UDCA and 7KLCA treatment for a further 512 h (8 full volume turnovers, 14 days) from 3-stage continuous culture fermentation systems. Graphs show Eubacterium rectale (Erec482), Roseburia spp. (Rrec584), Atopobium spp. (Ato291) and Clostridial cluster IX (Prop853). Graphs show bacterial numbers in log 10 (CFU/mL). Error bars are SEM.

FIG. 6 is a series of graphs showing the results of flow-FISH analysis of Example 2 from baseline samples after the first steady state (SS1) was reached after 512 h (8 full volume turnovers, 14 days) and after UDCA and 7KLCA treatment for a further 512 h (8 full volume turnovers, 14 days) from 3-stage continuous culture fermentation systems. Graphs show Faecalibacterium prausnitzii (Fprau655), Desulfovibrionales (DSV687), Clostridium histolyticum (Chis150) and Streptococcus spp. (Strp). Graphs show bacterial numbers in log 10 (CFU/mL). Error bars are SEM.

FIG. 7A-7E is a series of graphs showing the concentration of bacterial metabolites lactate (7A) and single chain fatty acids acetate (7B), butyrate (7C), propionate (7D) and valerate (7E), which were detected by GC-MS after the first steady state (SS1) and after UDCA and 7KLCA treatment. Error bars are mean standard error (SEM).

FIG. 8 is a comparison of two 1H NMR spectra of a sample taken from V3 (distal colon) before (upper trace) and after (lower trace) treatment with 7-KLCA with peaks relating to bacterial metabolites labelled (see Example 2). FIG. 8A shows the region from 2.5 ppm to 0 ppm and FIG. 8B shows the region from 4.0 ppm to 2.5 ppm.

FIG. 9 is a schematic illustration of the study design for Example 3 showing the diets of the mice in the test and control groups.

FIG. 10 is a plot showing the change in body weight (expressed in g) for the mice in the normal diet (CTRL 1), normal diet+7-KLCA (CTRL 2), HGD diet (CTRL 3) and HGD diet+7-KLCA (Treatment) groups of Example 3 evaluated from day 0 to day 60 (experimental end point). Data are presented as means±S.D. of n=5 mice for group.

FIG. 11 is a plot showing the blood glucose levels for the for the mice in the normal diet (CTRL 1), normal diet+7-KLCA (CTRL 2), HGD diet (CTRL 3) and HGD diet+7-KLCA (Treatment) groups of Example 3 at day 60. Values expressed in mg/dl are representative of n=5 for each group.

FIG. 12 is a plot showing the levels of HDL for the mice in the normal diet (CTRL 1), normal diet+7-KLCA (CTRL 2), HGD diet (CTRL 3) and HGD diet+7-KLCA (Treatment) groups of Example 3 at day 60. Values are expressed in mg/dl are representative of n=5 for group.

FIG. 13 is a plot showing the levels of LDL for the mice in the normal diet (CTRL 1), normal diet+7-KLCA (CTRL 2), HGD diet (CTRL 3) and HGD diet+7-KLCA (Treatment) groups of Example 3 at day 60. Values expressed in mg/dl are representative of n=5 for group.

FIG. 14 is a series of plots showing levels of total cholesterol for the mice in the normal diet (CTRL 1), normal diet+7-KLCA (CTRL 2), HGD diet (CTRL 3) and HGD diet+7-KLCA (Treatment) groups of Example 3 at day 60. Values expressed in mg/dl are representative of n=5 for group.

FIG. 15 is a Krona chart, displaying abundance and hierarchy simultaneously of DNA extracted from samples before 7-KLCA treatment. The Krona chart features a colour gradient signifying average e-values of BLAST hits within each taxon.

FIG. 16 is a Krona chart displaying abundance and hierarchy simultaneously of DNA extracted from samples after 7-KLCA treatment in the proximal colon (V1).

FIG. 17 is a Krona chart displaying abundance and hierarchy simultaneously of DNA extracted from samples after 7-KLCA treatment in the transverse colon (V2).

FIG. 18 is a Krona chart displaying abundance and hierarchy simultaneously of DNA extracted from samples after 7-KLCA treatment in the distal colon (V3).

### EXAMPLES

### Example 1—Batch Culture Fermentations

Fresh faecal samples were collected 1 h prior to inoculation from healthy adult male and female donors, who had not taken antibiotics for 3 months beforehand and had no history of gastrointestinal disorders. The faecal slurry was prepared by diluting the stool in phosphate saline buffer (PBS, pH 7.0) at a ratio of 1:10 and mixed in a stomacher for 2 min.

The method used was as described by Olano-Martin et al. (2000). 9 mL of autoclaved nutrient medium was added to 10 mL working volume fermentation vessels. The nutrient medium contained (g/L⁻¹): peptone water, 2; yeast extract, 2; NaCl, 0.1; K₂HPO₄, 0.04; KH₂PO₄, 0.04; MgSO₄·7H₂O, 0.01; CaCl₂)·6H₂O, 0.01; NaHCO₃, 2; hemin (dissolved in a few drops of 1 mol 1.1 NaOH), 0.05; cysteine HCl, 0.5; bile salts, 0.5; Tween® 80, 2 and 10 μL vitamin K topped up to 1 L with ddH₂O. The medium was flushed with gaseous N₂ overnight to create anaerobic conditions. Vessels were maintained at 37° C. via a circulating water bath and pH was maintained between 6.7 and 6.9 to mimic conditions in the distal colon, using a pH controller connected to 0.25 M solutions of HCl and NaOH. Immediately prior to faecal sample inoculation, 0.1 g of maltodextrin (1%, w/v) and 5 mg of 7-KLCA (0.5 mg/mL, w/v) were added to the vessels before adding 1 mL (v/v) faecal slurry. Samples were removed from the fermenters immediately after inoculation of the slurry (0 h) and at 5 h, 10 h and 24 h for enumeration of bacteria and metabolite analyses.

### Enumeration of Bacteria by Flow Cytometry-Fluorescent In Situ Hybridisation (Flow-FISH)

Samples (750 μL) were removed from in vitro fermentation vessels at 0, 5, 10 and 24 h and immediately placed on ice, before centrifugation at 13,000×g for 5 min and the supernatant discarded. Pelleted bacteria were fixed for 4 h at 4° C. in PBS and 4% (w/v) filtered paraformaldehyde (PFA, pH 7.2) in a ratio of 1:3 (v/v). Samples were washed twice with filtered PBS and resuspended in 300 μL of PBS and ethanol (1:1, v/v) then stored at −20° C. for up to 3 months.

Hybridisation was carried out using a method as described by Rigottier-Gois et al. (2003), using genus and group specific 16S rRNA-targeted oligonucleotide probes. Primers used were Non-Eub, Eub338, Bif164, Lab158, Bac303, Erec482, Rrec584, Ato291, Prop853, Fprau655, DSV687, and Strc493, which are shown in Table 1 below. Samples were screened using a flow cytometer (Accuri™ C6, BD Biosciences, USA) with Accuri™ CFlow software.

TABLE 1
Fluorescent in situ hybridisation oligonucleotide probe sequences (50 ng/uL)

| Probe | | | |
|---|---|---|---|
| Name | Sequence (5′ to 3′) | Target group(s) | Reference |
| Non-Eub | ACTCCTACGGGAGGCAGC | Control probe | Wallner |
| | (SEQ ID NO: 1) | complementary to | (1993) |
| | | EUB338. Background | |
| | | fluorescence and non- | |
| | | specific binding | |
| EUB338 | GCTGCCTCCCGTAGGAGT | Total bacteria | Amann |
| EUB33811 | (SEQ ID NO: 2) | | (1990), |
| EUB338III | GCAGCCACCCGTAGGTGT | | Daims |
| | (SEQ ID NO: 3) | | (1999) |
| | GCTGCCACCCGTAGGTGT | | |
| | (SEQ ID NO: 4) | | |
| Bif164 | CATCCGGCATTACCACCC (SEQ | Bifidobacterium spp | Langendijk |
| | ID NO: 5) | | (1995) |
| Lab158 | GGTATTAGCAYCTGTTTCAA | Lactobacillus and | Harmsen |
| | (SEQ ID NO: 6) | Enterococcus | (1999) |
| Bac303 | CCAATGTGGGGGACCTT (SEQ | Bacteriodes-Prevotella | Manz |
| | ID NO: 7) | | (1996) |
| Erec482 | GCTTCTTAGTCARGTACCG | Most of the Clostridium | Franks |
| | (SEQ ID NO: 8) | coccoides-Eubacterium | (1998) |
| | | rectale group | |
| | | (Clostridium subclusters | |
| | | XIVa and XIVb) | |
| Rrec584 | TCAGACTTGCCGYACCGC | Roseburia genus | Walker |
| | (SEQ ID NO: 9) | | (2005) |
| Ato291 | GGTCGGTCTCTCAACCC (SEQ | Atopobium- | Harmsen |
| | ID NO: 10) | Coriobacterium | (2000) |
| Prop853 | ATTGCGTTAACTCCGGCAC | Clostridial cluster IX | Walker |
| | (SEQ ID NO: 11) | | (2005) |
| Fprau655 | CGCCTACCTCTGCACTAC (SEQ | Faecalibacterium | Suau |
| | ID NO: 12) | prausnitzii | (2001) |
| DSV687 | TACGGATTTCACTCCT (SEQ ID | Desulfovibrio genus | Ramsing |
| | NO: 13) | (Dessulfovibrionales and | (1996) |
| | | Dessulfuromonadales) | |
| Chis150 | TTATGCGGTATTAATCTYCCTTT | Most of the Clostridium | Franks |
| | (SEQ ID NO: 14) | histolyticum group | (1998) |
| | | (Clostridium clusters I | |
| | | and II) | |
| Strc493 | GTTAGCCGTCCCTTTCTGG | Streptococcus spp. and | Franks |
| | (SEQ ID NO: 15) | some Lactococcus spp. | (1998) |

R represents G/A
Y represents T/C

Once defrosted, samples were then vortexed for 10 s and 75 μL of the sample added to 500 μL of PBS in a 1.5 mL tube to be vortexed again and centrifuged at 13,000×g for 3 min. The supernatant was removed and discarded. 100 μL Tris-EDTA buffer containing lysozyme (1 mg/mL) was added to the tube, mixed using a pipette, then incubated in the dark for 10 min. Samples were vortexed and centrifuged at 13,000×g for 3 min and the supernatant removed. 500 μL of PBS was added to the tube, the pellet resuspended using a pipette then vortexed and centrifuged at 13,000×g for 3 mins. Supernatant was removed and pellets resuspended in 150 μL of hybridisation buffer (0.9 M NaCl, 0.2 M Tris-HCl (pH 8.0), 0.01% sodium dodecyl sulphate, 30% formamide), vortexed and centrifuged at 13,000×g for 3 min. Supernatant was again removed and pellets resuspended in 1 mL hybridisation buffer. 4 μL of oligonucleotide probe solutions (50 ng/L) were added to a 1.5 mL centrifuge tube with 50 μL of the sample, vortexed and incubated at 36° C. overnight.

Once hybridisation had been completed, 125 μL of hybridisation buffer was added to each tube, vortexed, centrifuged at 13,000×g for 3 mins and the supernatant carefully removed. 175 μL of washing buffer (0.064 M NaCl, 0.02 M Tris/HCl (pH 8.0), 0.5 M EDTA (pH 8.0), 0.01% sodium dodecyl sulphate) kept at 40° C. was added to each tube, resuspending the pellets. The tubes were then vortexed and incubated for 20 min at 38° C. in the dark, to remove non-specific binding of primers. Samples were then centrifuged at 13,000×g for 3 min, the supernatant removed, 300 μL PBS added, then vortexed. Samples were kept refrigerated at 4° C. in the dark prior to flow cytometry analysis.

### Summary of Flow-FISH

These batch cultures focussed on the effects of 7-KLCA on bacterial population numbers. Samples were taken at 0, 5, 10 and 24 hours for flow-FISH analysis, which revealed changes in bacterial composition under different 7-KLCA concentrations. Three donors were used in these fermentations, with vessels containing either 7-KLCA and maltodextrin (as a carbon source), or maltodextrin (as a carbon source) alone.

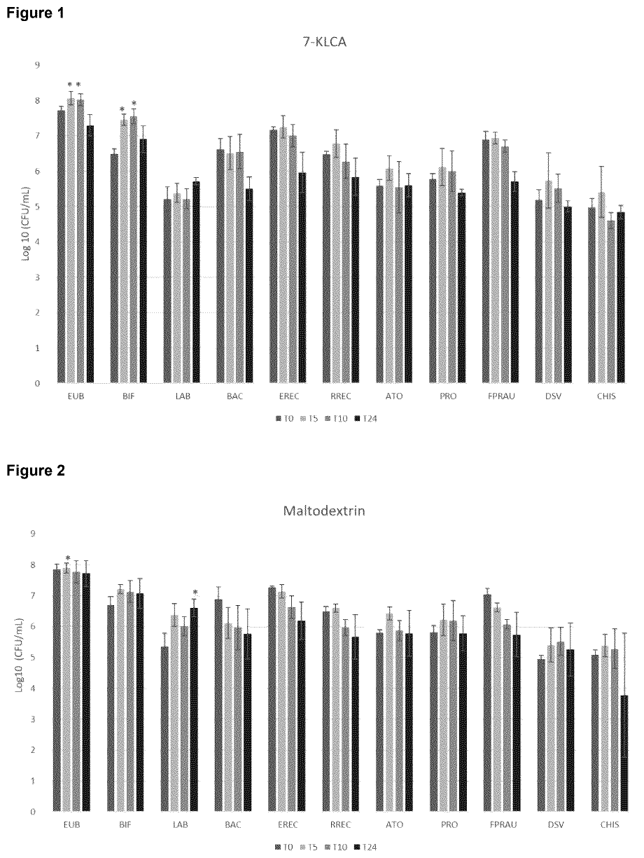

The results are shown in Table 2 and in FIGS. 1 and 2. Analysis revealed that there were statistically significant increases in total bacteria (EUB in FIGS. 1 and 2, Total in Table 1) and Bifidobacterium spp. (BIF) at 5 and 10 h in vessels containing 7-KLCA. Bifidobacterium numbers increased from Log10 6.48±0.14 CFU/mL to Log10 7.45±0.16 CFU/mL at T5 and 5 to Log10 7.55±0.21 CFU/mL at T10.

Meanwhile, there were no significant increases in Bifidobacterium numbers in the maltodextrin only vessel, only in total bacteria and Lactobacillus (LAC) at 5 h.

There were also no increases in pathogenic bacterial species, including Atopobium, Coriobacterium, Clostridium histolyticum, Clostridium perfringens, Desulfovibrionales and Desulfuromonadales, in either vessels containing 7-KLCA or the control vessels.

TABLE 2

| | | BIF | LAB | BAC | EREC | RREC | ATO | PROP | FPRAU | DSV | CHIS | Total |
|---|---|---|---|---|---|---|---|---|---|---|---|---|
| | | Log 10 | Log 10 | Log 10 | Log 10 | Log 10 | Log 10 | Log 10 | Log 10 | Log 10 | Log 10 | Log 10 |
| | Time | Bacteria/ | Bacteria/ | Bacteria/ | Bacteria/ | Bacteria/ | Bacteria/ | Bacteria/ | Bacteria/ | Bacteria/ | Bacteria/ | Bacteria/ |
| | (h) | mL (s.e.) | ml (s.e.) | mL (s.e.) | mL (s.e.) | mL (s.e.) | mL (s.e.) | mL (s.e.) | mL (s.e.) | mL (s.e.) | mL (s.e.) | mL (s.e.) |
| Negative | 0 | 6.42 | 5.140 | 6.304 | 7.277 | 6.282 | 5.812 | 6.310 | 6.740 | 5.018 | 5.718 | 7.738 |
| | | (0.54) | (0.26) | (0.23) | (0.14) | (0.10) | (0.16) | (0.21) | (0.26) | (0.07) | (0.20 | (0.15) |
| | 5 | 6.243 | 5.433 | 5.765 | 6.454 | 5.652 | 6.015 | 6.380 | 5.968 | 4.274 | 5.478 | 7.370 |
| | | (0.64) | (0.16) | (0.80) | (0.55) | (0.31) | (0.07) | (0.17) | (0.73) | (0.39) | (0.57) | (0.39) |
| | 10 | 5.294 | 4.459 | 4.650 | 5.050 | 2.857 | 5.229 | 2.392 | 3.086 | 4.117 | 4.030 | 6.458 |
| | | (0.76) | (0.13) | (0.56) | (0.75) | (1.65) | (0.44) | (1.38) | (1.77) | (0.24) | (0.16) | (0.52) |
| | 24 | 3.248 | 4.769 | 4.601 | 2.924 | 4.468 | 4.783 | 2.523 | 2.856 | 2.577 | 4.445 | 6.699 |
| | | (1.88) | (0.56) | (0.44) | (1.69) | (0.42) | (0.54) | (1.45) | (1.65) | (1.49) | (0.35) | (0.37) |
| Malto- | 0 | 6.694 | 5.346 | 6.889 | 7.266 | 6.495 | 5.789 | 5.807 | 7.040 | 4.934 | 5.085 | 7.850 |
| dextrin | | (0.26) | (0.43) | (0.39) | (0.04) | (0.15) | (0.10) | (0.22) | (0.18) | (0.14) | (0.16) | (0.18) |
| | 5 | 7.212 | 6.368 | 6.106 | 7.136 | 6.595 | 6.417 | 6.211 | 6.613 | 5.398 | 5.379 | 7.889 |
| | | (0.15) | (0.37) | (0.51) | (0.23) | (0.14) | (0.22) | (0.52) | (0.14) | (0.56) | (0.36 ) | (0.16) |
| | 10 | 7.124 | 6.007 | 5.959 | 6.623 | 5.957 | 5.865 | 6.182 | 6.066 | 5.514 | 5.272 | 7.773 |
| | | (0.35) | (0.31) | (0.71) | (0.37) | (0.26) | (0.33) | (0.65) | (0.15) | (0.45) | (0.64) | (0.36) |
| | 24 | 7.067 | 6.597 | 5.758 | 6.180 | 5.658 | 5.770 | 5.781 | 5.732 | 5.252 | 3.760 | 7.714 |
| | | (0.48) | (0.28) | (0.82) | (0.62) | (0.72) | (0.74) | (0.57) | (0.72) | (0.86) | (2.01) | (0.11) |
| 7-KLCA | 0 | 6.485 | 5.203 | 6.608 | 7.163 | 6.471 | 5.581 | 5.780 | 6.896 | 5.185 | 4.967 | 7.714 |
| | | (0.14) | (0.36) | (0.31) | (0.08) | (0.09) | (0.17) | (0.14) | (0.23) | (0.29) | (0.25) | (0.11) |
| | 5 | 7.452 | 5.380 | 6.505 | 7.251 | 6.773 | 6.082 | 6.116 | 6.931 | 5.728 | 5.407 | 8.057 |
| | | (0.16) | (0.27) | (0.47) | (0.32) | (0.39) | (0.35) | (0.53) | (0.17) | (0.78) | (0.72) | (0.19) |
| | 10 | 7.548 | 5.214 | 6.547 | 7.000 | 6.276 | 5.540 | 5.993 | 6.702 | 5.513 | 4.596 | 8.014 |
| | | (0.21) | (0.29) | (0.49) | (0.32) | (0.49) | (0.73) | (0.58) | (0.17) | (0.40) | (0.22) | (0.17) |
| | 24 | 6.901 | 5.711 | 5.494 | 5.956 | 5.831 | 5.599 | 5.394 | 5.698 | 4.995 | 4.841 | 7.290 |
| | | (0.38) | (0.10) | (0.34) | (0.57) | (0.53) | (0.33) | (0.10) | (0.28) | (0.16) | (0.20) | (0.30) |

Bacteria Covered (see Table 1)
BIF-Bifidobacterium species
LAB-Lactobacillus-Enterococcus
BAC-Bacteriodes-Prevotella
EREC-Eubacterium rectale-Clostridium coccoides
RREC-Roseburia and Eubacterium rectale
ATO-Atopobium-Coriobacterium
PROP-Clostridial cluster IX
FPRAU-Faecalibacterium prausnitzii;
DSV-Desulfovibrionales and Dessulfuromonadales
CHIS-Clostridium histolyticum-perfringens

### Gas Chromatography Mass Spectroscopy (GC-MS) for Short Chain Fatty Acid (SCFA) and Lactate Analysis

1.5 mL samples from vessels containing 7-KLCA+maltodextrin and those containing maltodextrin alone were taken at 0, 5, 10 and 24 h and centrifuged at 13,000×g for 10 min and supernatants stored at −20° C. until needed for metabolite analysis. Once the samples had been removed from −20° C. storage and had defrosted, these were vortexed and centrifuged again at 13,000×g for 10 min. 1 mL aliquots of sample supernatant were then transferred into flat-bottomed glass tubes with 50 μL internal standard solution (0.1 M 2-ethylbutyric acid). 500 μL of concentrated hydrochloric acid (HCl) and 3 mL of diethyl ether were added to the sample in each glass tube, which was then vortexed for 1 min and centrifuged at 2000×g for 10 min. 400 μL of pooled ether extract was then added to 50 μL N-(tert-butyldimethylsilyl)-N-methyltrifluoroacetamide (MTBSTFA) in a GC screw-cap vial. External samples of 1 M acetic, butyric, propionic and lactic acid were also prepared using Richardson SCFA derivatisation method. Samples were left at room temperature for 72 h to allow metabolites to completely derivatise. An Agilent/HP 6890 Gas Chromatograph (Hewlett Packard, UK) fitted with a HP-5 MS 30 m×0.25 mm column with a 0.25 μm coating (Crosslinked (5%-Phenyl)-methylpolysiloxane, Hewlett Packard, UK) was used for metabolite analysis. Quantification of the samples was obtained through calibration curves of lactic, acetic, propionic, butyric acids at concentrations between 6.25 and 100 mM from the external standards.

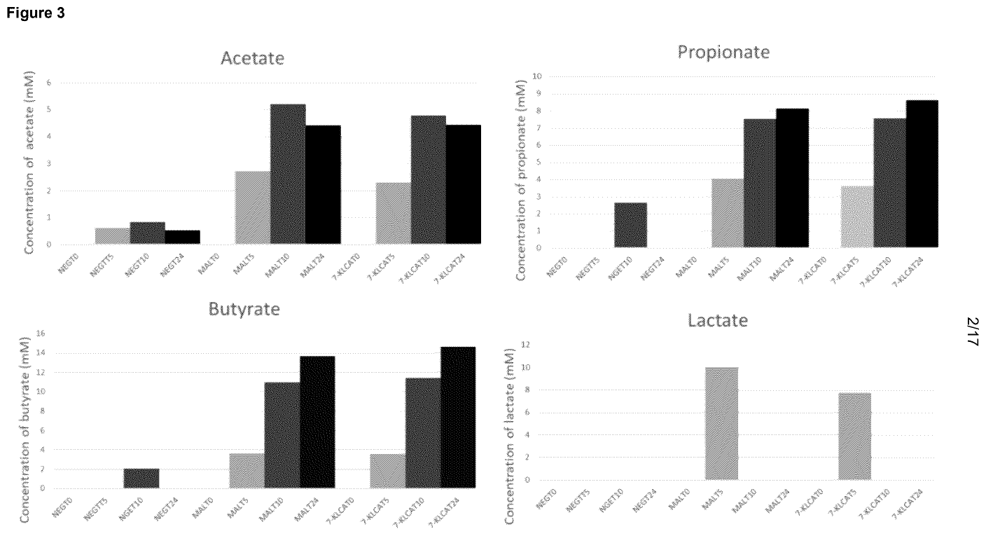

The preliminary results from one donor show there are differences in metabolic profiles for different 7-KLCA concentrations, promoting beneficial SCFA production that would be supportive of conferring a health benefit (FIG. 3).

### Example 2-3—Stage Gut Models

3-stage gut models are continuous systems that are designed to reproduce the nutritional, temporal and physiochemical features of microbiota within the large intestine. The 3-stage gut models used in the present examples were developed at the University of Reading and have been validated via the chemical and microbiological measurements on intestinal contents of sudden death victims and are excellent models to run, as an indicator of how test substrates would act in vivo. These experiments take longer than the experiment described in Example 1 as conditions prior to inoculation with a test compound, requiring several turnovers in order to reach a steady state, which is important for understanding whether any changes detected are as a result of the test substrate added. Visualisation of bacterial populations within gut models can be performed using flow FISH cytometry and 16S rRNA sequencing analysis, which when coupled to mass spectrometry can reveal changes in bacterial communities and metabolism.

The objective of the 3-stage gut model experiments was to establish the effects of 7KLCA on the gut microbiome of healthy volunteers, using in vitro gut fermentation and downstream analysis of bacterial populations and resulting metabolites, using flow-FISH cytometry, 16S rRNA sequencing, gas chromatography-mass spectrometry and NMR spectroscopy.

### 3-Stage Gut Models

In this example, the test substance was 7-KLCA and UDCA was used as a control.

A three-stage continuous culture system was set up to mimic the defining sections of the large intestine, the proximal colon, transverse colon and the distal colon. Vessels were set up in sequence, and simulated nutritional, temporal and physiochemical features of microbiota within the proximal (V1, 80 mL, pH=5.5), transverse (V2, 100 mL, pH=6.2) and distal colon (V3, 120 mL, pH=6.8). The system was maintained in anaerobic conditions by sparging the vessels with nitrogen gas (15 mL/min) and kept at a continuous temperature of 37° C. via a circulating water bath.

The vessels were inoculated with faecal slurry at a concentration of 6% in each of the three vessels; samples were donated from 3 healthy adult donors. The faecal donors had not taken antibiotics within 6 months of the experiment and were not regular consumers of prebiotic or probiotic supplements. Donors collected faecal samples in anaerobic jars (AnaeroJar™ 2.5L, Basingstoke, UK, Oxoid Ltd.) and stored under anaerobic conditions with the use of anaerobic sachets (AnaeroGen, Oxoid) and used within 1 h of production. The faecal slurry was prepared at a 1 in 5 (w:v) stool to PBS (anaerobic phosphate buffered saline; 0.1 mol/L; pH 7.4) and was homogenised using a stomacher (Stomacher 400, Seward, West Sussex, UK) for 2 min (240 paddle beats/min).

Samples were collected from V1, 2, 3 immediately upon inoculation of the fermentation systems (T-1) and 24 hours post-inoculation (T-0). The T0 samples provide ideal starting conditions for the continuous culture fermentation, as the bacterial population numbers would have increased and settled to the species composition that are found in the conditions in each of the vessels and to the pH conditions. Upon taking T0 samples, the three-stage continuous culture system began the first steady state 1 (SS1) and the flow of nutrient media was started with a retention time appropriate to heathy adults when considering the operating volume (300 mL) and retention time (flow rate 6 mL/h) of the gut model system.

The first steady state (SS1), when equilibrium was reached, was after 512 h (8 full volume turnovers, equivalent to 14 days) using standard gut model media. Once equilibrium was reached, daily dosing of the bile acids (400 mg/day) was given to V1, and the gut model entered the second steady state (SS2) phase. Again, standard gut model media was used and given at the same flow rate, with the bile acid being dosed to V1 at the same time every day during SS2 (512 h (8 full volume turnovers, 14 days). Samples were collected two and one day(s) (SS1-2 and SS1-1) prior to the SS1 sample collection to ensure the gut model system had reached equilibrium (using GC-MS). A sample was also collected from each vessel in the system after the second steady state (SS2).

### Enumeration of Bacteria by Flow Cytometry-Fluorescent In Situ Hybridisation (Flow-FISH)

This was carried out by the same method and using the same 16S rRNA-targeted oligonucleotide probes.

### Gas Chromatography Mass Spectroscopy (GC-MS) for Short Chain Fatty Acid (SCFA) and Lactate Analysis

1.5 mL samples from each vessel were taken at 0, 5, 10 and 24 h and centrifuged at 13,000× g for 10 min and supernatants stored at −20° C. until needed for metabolite analysis. Samples were prepared for GC-MS using the SCFA derivatisation method as described by Richardson et al. (1989). Once the samples had been removed from −20° C. storage and had defrosted they were vortexed and centrifuged again at 13,000× g for 10 min. 1 mL aliquots of sample supernatant were then transferred into flat-bottomed glass tubes with 50 μL internal standard solution (0.1 M 2-ethylbutyric acid). 500 μL of concentrated hydrochloric acid (HCl) and 3 mL of diethyl ether were added to the sample in each glass tube, which was then vortexed for 1 min and centrifuged at 2000×g for 10 min. 400 μL of pooled ether extract was then added to 50 μL N-(tert-butyldimethylsilyl)-N-methyltrifluoroacetamide (MTBSTFA) in a GC screw-cap vial. External samples of 1 M acetic, butyric, propionic and lactic acid were also prepared using a SCFA derivatisation method (Richardson et al., 1989). Samples were left at room temperature for 72 h to allow metabolites to completely derivatise. An Agilent/HP 6890 Gas Chromatograph (Hewlett Packard, UK) fitted with a HP-5 MS 30 m×0.25 mm column with a 0.25 μm coating (Crosslinked (5%-Phenyl)-methylpolysiloxane, Hewlett Packard, UK) was used for metabolite analysis. Quantification of the samples was obtained through calibration curves of lactic, acetic, propionic, butyric acids at concentrations between 6.25 and 100 mM from the external standards.

DNA Extraction for 16S rRNA Sequencing

1.5 mL samples from batch culture fermentations were taken at each time sampling point and centrifuged at 13,000×g for 10 mins. The pellet was then stored at −20° C. until needed for DNA extraction. A QIAamp® PowerFecal® Pro DNA Kit supplied by QIAGEN® was used for extraction. The pelleted samples were defrosted on ice, then 800 μL of Solution CD1 was added to the pellet and homogenised. Each mix was then pipetted into a PowerBead Pro Tube and vortexed briefly to mix. The samples were then homogenised by loading the PowerBead Pro Tubes using a TissueLyser II set to 30 Hz for 2 min before placing on ice for 3 mins, followed by another 2 mins on the TissueLyser II. The tubes were then centrifuged at 15,000×g for 1 min, before the supernatant was then transferred to a clean 2 ml microcentrifuge tube. 200 μL of Solution CD2 was added to the tube, which was vortexed for 5 s, then centrifuged at 15,000×g for 1 min. Up to 700 μL of the supernatant was 2 ml was transferred to a clean microcentrifuge tube and 600 μl of Solution CD3 added and vortexed for 5 s. All the lysate was then loaded onto an MB Spin Column and centrifuged at 15,000×g for 1 min so that the DNA is selectively bound to the silica membrane. The MB Spin Column was then placed into a clean 2 ml Collection Tube and 500 μl of Solution EA added before centrifuging at 15,000×g for 1 min. The flow-through was then discarded and the MB Spin Column back into the same 2 ml Collection Tube. 500 μl of Solution C5 was then added to the MB Spin Column and centrifuged at 15,000×g for 1 min. The flow-through was then discarded and the MB Spin Column placed into a new 2 ml Collection Tube. The tubes were then centrifuged at 16,000×g for 2 min. The column was then placed into a new 1.5 ml Elution Tube and 50 μL of Solution C6 pipetted onto the centre of the white filter membrane. The quality of the DNA was then analysed on a NanoDrop™ before the samples were frozen at −80° C. before sending to Microsynth AG, Switzerland for 16S rRNA sequencing.

### NMR Sample Preparation

Samples were defrosted and vortexed for 10s to resuspend the particles, before allowing them to settle again in the tubes. 400 μL of the sample (avoiding the pellet) was then transferred into a new 1.5 mL centrifuge tube containing 200 μL of 0.2M sodium phosphate buffer solution (pH7.4) made in 100% deuterium oxide (D₂O), which also contained 0.01% of sodium 3-(trimethylsilyl) [2, 2, 3, 3,-2H4] propionate (TSP) and 3 mM sodium azide (NaN₃). D₂O is required for the field lock of the NMR spectrometer, and TSP as an internal reference standard for calibration of acquired spectral profiles. The mixture was vortexed and centrifuged for 10s, and 550 μL of the mixture added to an NMR tube with an outer diameter of 5 mm. Samples were analysed in a randomised order, and one-dimensional spectroscopic data acquired, according to established metabolic profiling protocols using standard one-dimensional NMR pulse sequence with water pre-saturation.

### Results

### Bacterial Enumeration by Flow-FISH Cytometry

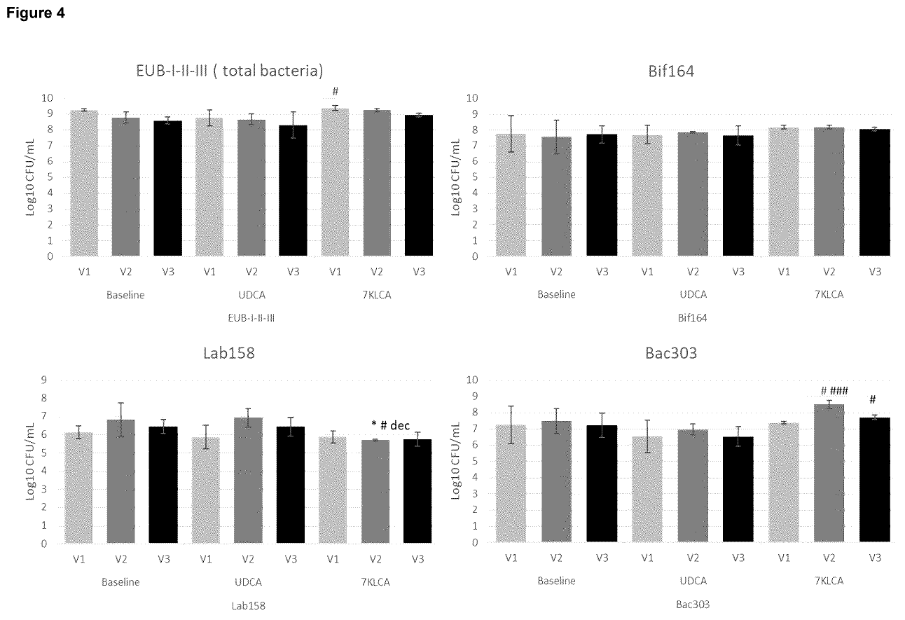

As shown in FIG. 4, one-way ANOVA analysis, using IBM® SPSS® Statistics, revealed that there were statistically significant differences between total bacteria (EUB-I-II-III) of the 7-KLCA treated gut models (Log10 9.35±0.16 CFU/mL) and the UDCA treated models (Log10 8.74±0.50 CFU/mL) in the proximal region (V1). Despite no significant increases in total bacteria numbers from Baseline and after a bile acid treatment, numbers of bacteria were greater after 7-KLCA treatment in all three regions of the model.

Furthermore, one-way ANOVA analysis showed a statistical difference between the two treatments in the transverse region (V2) in Bacteroides-Prevotella (BAC303) population numbers (P<0.05), Log10 8.47±0.24 CFU/mL (7-KLCA) and Log10 6.94±0.33 CFU/mL (UDCA).

Paired sample t-test analysis showed statistically significant differences in Bacteroides-Prevotella population numbers in the distal region (V3) (P<0.05), Log10 7.69±0.15 CFU/mL (7-KLCA) and 6.51±0.60 CFU/mL (UDCA). Paired sample t-test analysis showed greater statistical differences in the transverse region (P<0.001) between the 7-KLCA treated gut models and the UDCA treated models.

Whilst there were no statistically significant increases in Bifidobacterium spp. (Bif164) between the Baseline population numbers and after 7-KLCA or UDCA treatment, there was an observed increase in bacterial numbers across all three stages of the gut model (V1, 2 and 3) after 7-KLCA treatment, with the greatest difference observed in the transverse region, with numbers increasing from Log 10 7.54±1.06 CFU/mL to Log 10 8.16±0.13 CFU/mL.

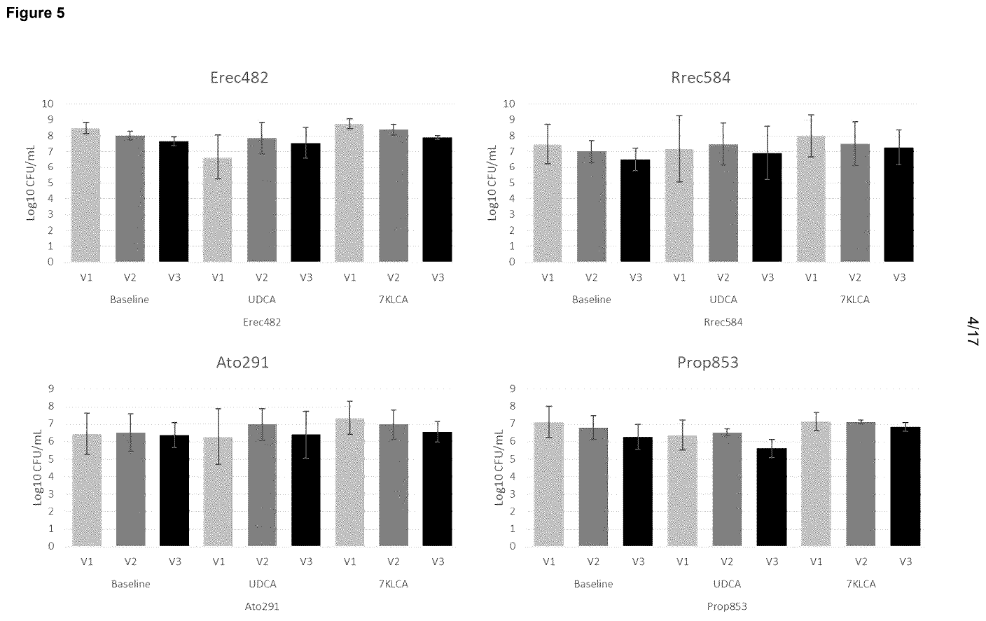

FIG. 5 shows that although there were no statistically significant increases, there was an increase in Eubacterium rectale and Roseburia spp. across V1, V2 and V3 between the baseline and after 7-KLCA treatment.

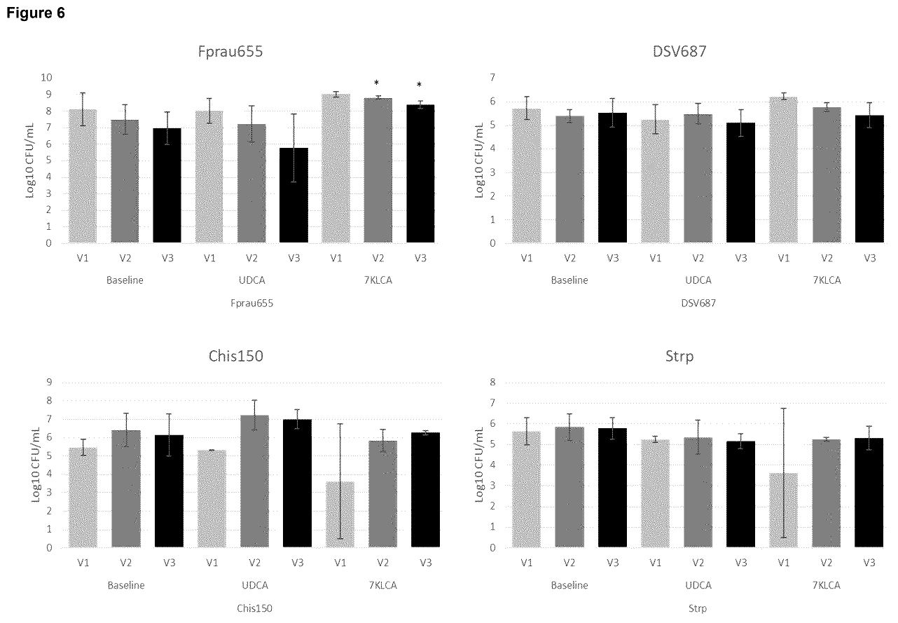

As shown in FIG. 6, there were statistically significant increases in numbers of butyrate-producing Faecalibacterium prausnitzii between the baseline and after 7KLCA treatment in V2 (Log10 7.47±0.91 CFU/mL to Log10 8.81±0.09 CFU/mL) and V3 (Log106.95±0.97 CFU/mL to Log10 8.39±0.22 CFU/mL). UDCA treatment did not lead to increases in Faecalibacterium prausnitzii.

FIG. 6 also shows that there were no statistically significant increases in groups pathogenic species, Clostridium histolyticum, Desulfovibrionales or Streptococcus with either UDCA or 7-KLCA.

These results demonstrate that, while both 7-KLCA and UDCA are able to increase the numbers of beneficial bacteria in the gut, 7-KLCA is more effective than UDCA at increasing numbers of total bacteria (EUB-I-II-III) in the proximal region. 7-KLCA also provides superior results to UDCA in increasing Bacteroides-Prevotella (BAC303) population numbers in the transverse and distal regions. 7-KLCA treatment also led to an increase in Bifidobacterium spp. (Bif164), Eubacterium rectale and Roseburia spp., although these results were not significant. There was no such increase following UDCA treatment.

The flow-FISH cytometry results show that while both 7-KLCA and UDCA are beneficial, 7-KLCA is superior to UDCA in increasing some beneficial bacteria.

### Bacterial Metabolite Quantification by GC-MS

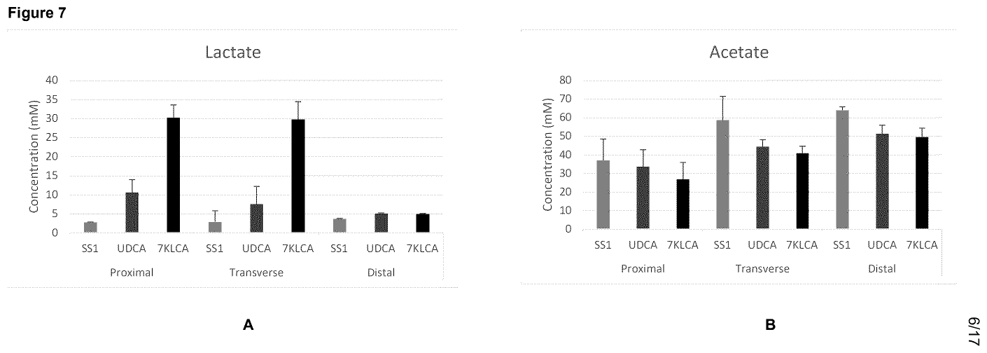

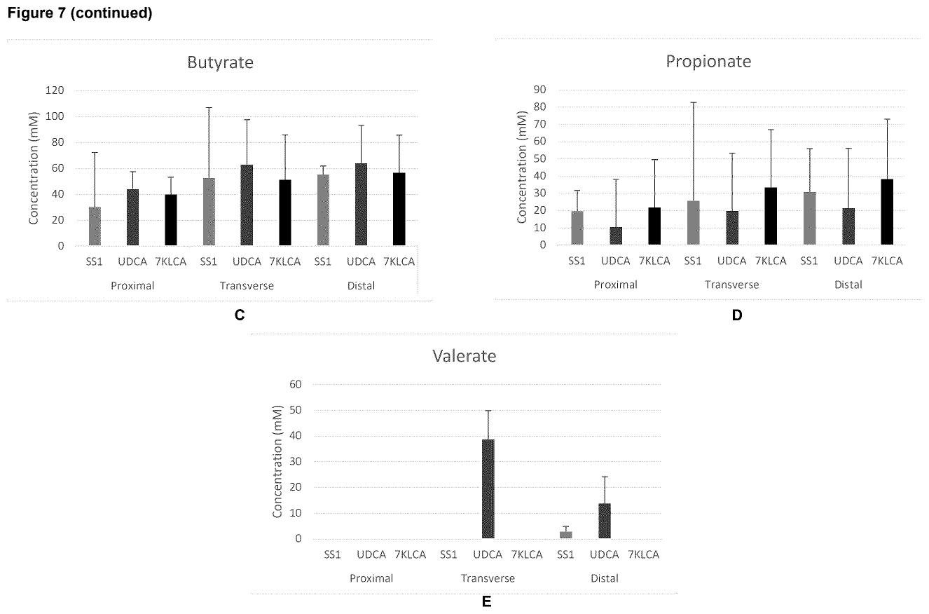

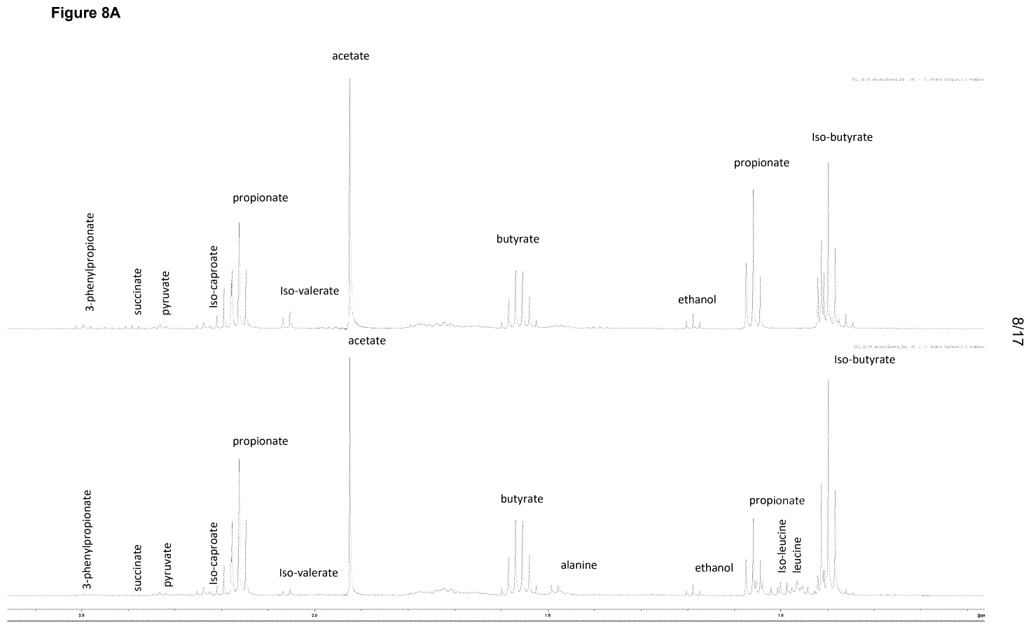

As shown in FIG. 7A, there were no statistically significant changes in lactate, however, there were increases measured in this metabolite after 7KLCA treatment in V1, V2 and V3.

FIGS. 7B, 7C, 7D and 7E show respectively that there were no statistically significant changes in acetate, butyrate, propionate or valerate concentration, however, there were increases measured in propionate concentration after 7KLCA treatment in V1, V2 and V3.

Bacterial Composition by 16s rRNA Sequencing

The abundance and hierarchy of DNA extracted from healthy in vitro gut samples was measured using 16s rRNA sequencing before and after 7-KLCA treatment.

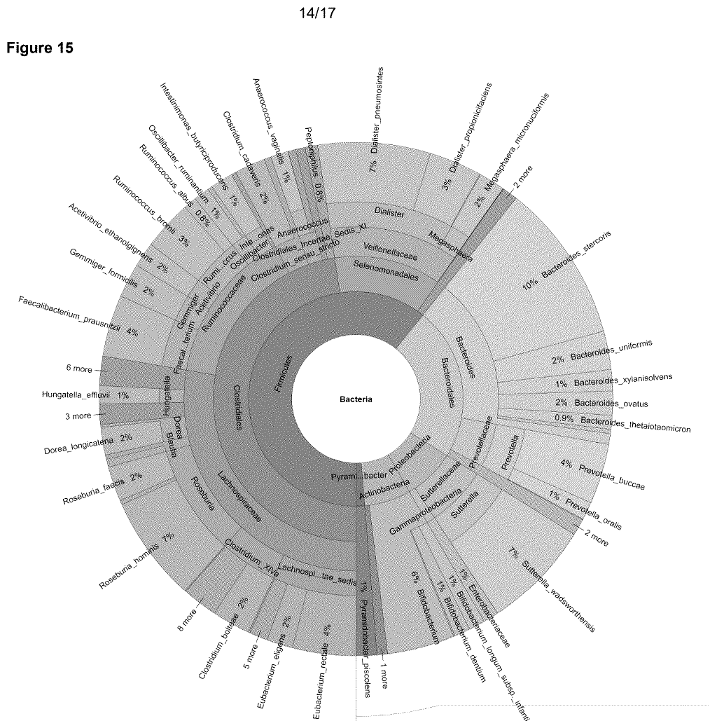

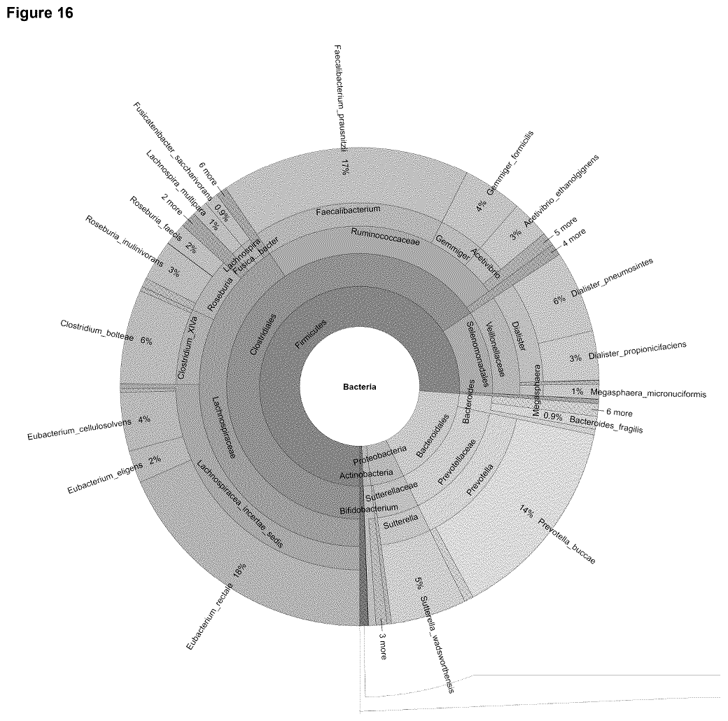

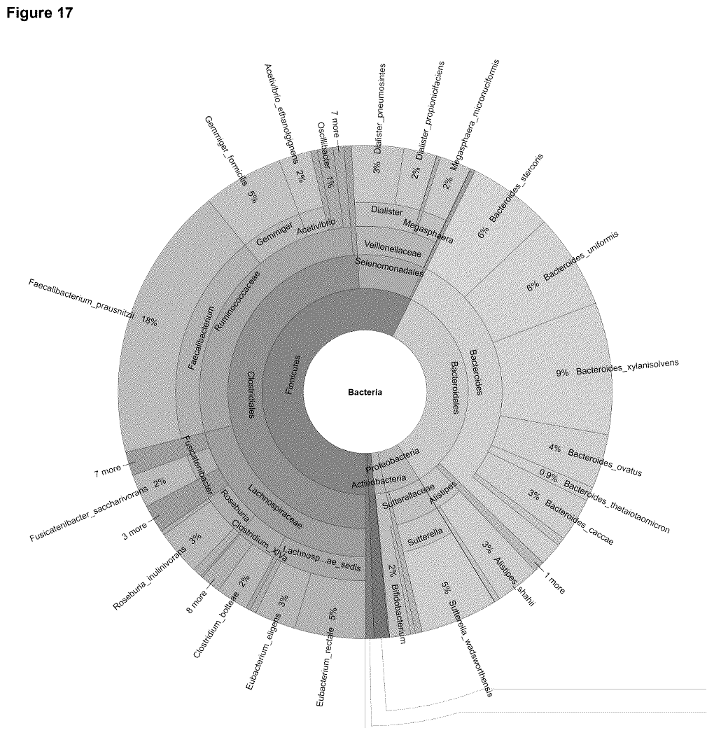

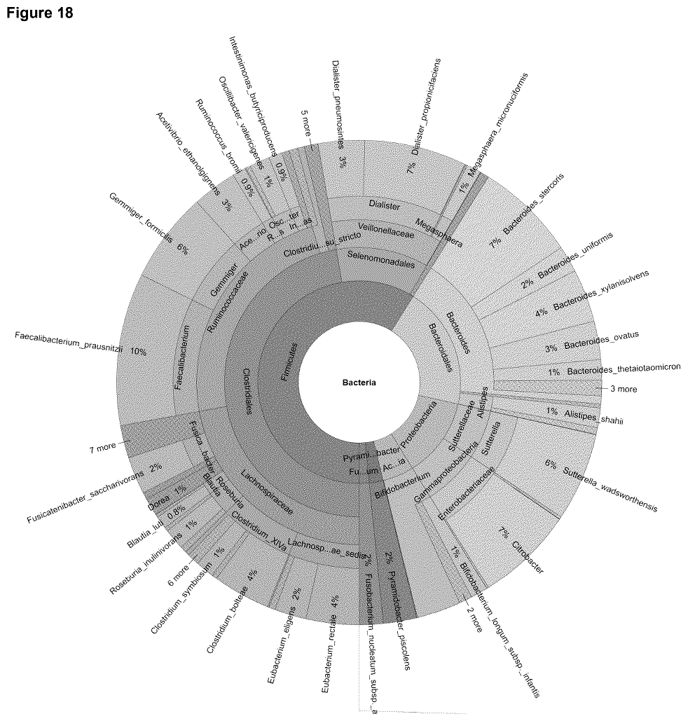

As can be seen from the Krona charts of FIGS. 15 to 18, before 7-KLCA treatment the percentage of probiotic species Faecalibacterium prausnitzii was 4%, Eubacterium rectale was also detected at 4%, Bacteroides xylanisolvens at 1% and Roseburia species at a combined 9%. Dialister pneumosintes has shown pathogenic potential in various sites of the body. Before 7-KLCA treatment, this bacterium made up 7% of total bacteria composition. After 7-KLCA treatment, this percentage dropped to 3% in both the transverse and distal colon (V2 and V3).

After 7-KLCA treatment, in the proximal region of the colon, the abundance of Faecalibacterium prausnitzii increases to 17% whereas Eubacterium rectale rises to 18%.

Similarly, in the transverse region of the colon, F. prausnitzii increases steeply, reaching 18% of total bacterial composition. Bacteroides xylanisolvens, which was measured at 2% before 7-KLCA treatment, increased to 9% after bile acid intervention. B. xylanisolvens, has been described as a New Generation Probiotic (NGP) due to its immune regulation properties and anti-cancer attributes (Ulsemer et al., 2016). Eubacterium eligens was recorded as 2% before 7-KLCA treatment, however this species, which has been found to hold anti-inflammatory activity via the promotion of IL-10 production by epithelial cells rises to 5% in V2 and remains 2% in V1 and V3.

After 7-KLCA treatment, F. prausnitzii increases, from 0.6% to 10% in V3 (distal colon). Bifidobacterium spp. are at 5%, which whilst lower than the baseline, indicates that the species can grow successfully with 7-KLCA treatment. Sequencing also revealed that Clostridium species decreased from 5.6% total bacterial species detected, to 4.5% after 7-KLCA treatment.

### Metabolite Spectra Analysis by ¹H NMR

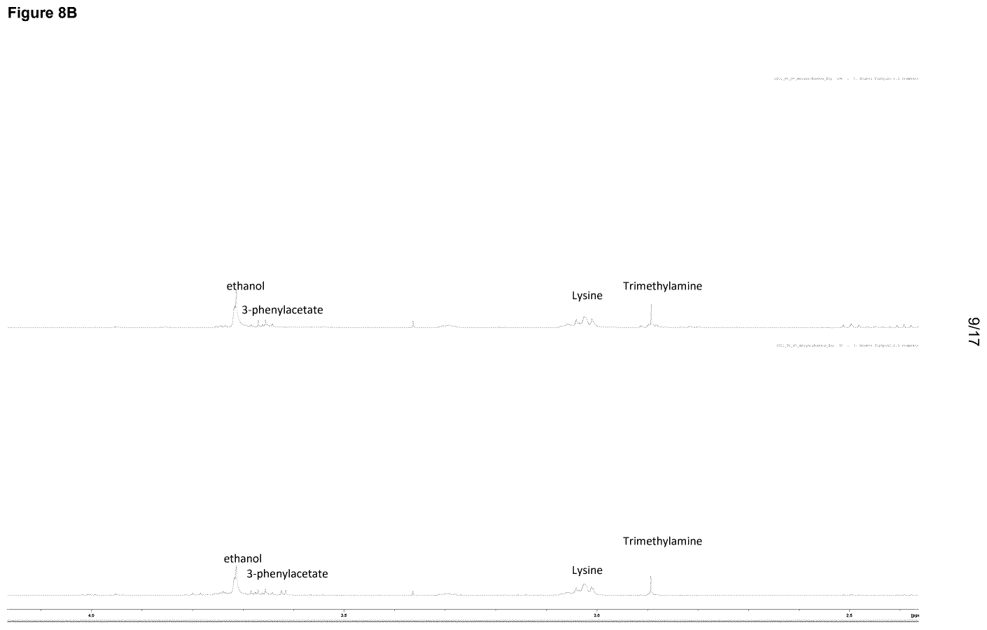

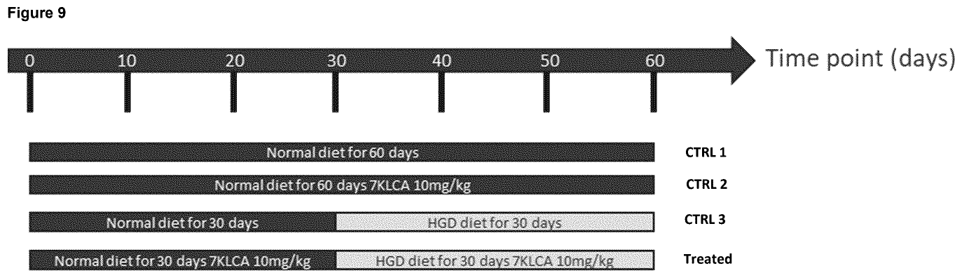

As shown in FIGS. 8A and 8B, 1H NMR spectral analysis before and after 7-KLCA treatment revealed the presence after treatment of bacterial metabolites iso-leucine and leucine, two amino acids that are important in immunity, protein metabolism, fatty acid metabolism and glucose transportation. Alanine also was detected after 7-KLCA treatment, a metabolite which is essential to protein synthesis and as an energy source for muscles and the central nervous system. After 7-KLCA treatment, peaks of butyrate were greater than the peaks of these metabolites prior to bile acid treatment. Butyrate is an important SCFA that helps maintain the barrier function of the gut wall, which can aid digestive health, help control inflammation and prevent disease.

¹H NMR spectral analysis also exposed the presence of several other bacterial metabolites, including propionate and acetate, both of which had higher peaks after 7-KLCA treatment. Other metabolites detected include: lysine, pyruvate, valerate and iso-valerate, all of which have important roles in the body including protein synthesis, energy metabolism and immune regulation.

### Example 3—Studies on Efficacy in Reducing Glycaemia in Mice

### Experimental Plan

The study was performed in the Laboratory of Department of Medicine and Surgery, University Federico II of Naples, Italy.

The study design:
CTRL 1 Mice on normal diet for 60 days (5 mice)
CTRL 2 Mice on normal diet with 7KLCA at 10 mg/kg for 60 days (5mice)
CTRL 3 Mice on normal diet for 30 days, then 30 days on a high glucose diet (5 mice)
Treated Mice on normal diet for 30 days, then 30 days on a high glucose diet with 7KLCA throughout the study (5 mice)

Standard diet (10.6% fat J/J, 29.0% protein J/J and 60.4% carbohydrate J/J). High glucose diet (10.5% fat J/J, 16.4% protein J/J and 73.1% carbohydrate J/J). For all experimental group, free accesses to tap water The animals were kept in group of 5 animals/sex/group/cage in Plexiglas cages.

Experiments were carried out in 8-12-week-old male CD-1 mice according to the guidelines for the safe use and care of experimental animals in accordance with the Italian D.L. no. 116 of 27 Jan. 1992 and associated guidelines in the European Communities Council (86/609/ECC and 2010/63/UE) including the 3Rs concept (Kilkenny et al, 2010; McGrath et al, 2015). Animals were housed with ad libitum access to food and water and maintained on a 12-hour light/dark cycle. Experimental study groups were randomized and blinded. The animals in the control groups were handled in an identical manner to the treated group using the respective vehicle and the same volume of administration. Tested item formulation or vehicle was administered by oral gavage. The body weight was recorded once before the assignment to the experimental groups, on the first day of administration, and then weekly during the treatment.

### Preparation of the Test Formulation

The dose of 7-KLCA used in this study was 10 mg/kg/day. The vehicle used was DMSO in saline solution (NaCl 0.9%), 1/3 w/w. 7-KLCA was solubilised in DMSO (1 part w/w) and then resuspended in saline solution (3 parts w/w). This solution (200 μl/mouse) was prepared freshly every day and was kept under magnetic stirring during the daily administration.

Test system

| | |
|---|---|
| Species/strain | Healthy CD-1 mice |
| Source | Charles River, Italy |
| Sex | Male |
| Age at the start of the treatment | approx. 8-12 weeks old |
| Body weight at the allocation of the animals | 26 g ± 2 g |
| to the experimental group | |

### Housing and Feeding Conditions

### Full Barrier in the Air-Conditioned Room

| | |
|---|---|
| Temperature | 22 ± 3° C. |
| Relative humidity | 55 ± 10% |
| Artificial light sequence | 12 hours light 12 hours dark |
| Air change | 10x/hour |

### Body Weight

The body weight was recorded once before the assignment to the experimental groups, on the first day of administration, and then weekly.

### Clinical Observation

All animals were observed for clinical signs during the entire treatment period of 60 days. General clinical observations were performed at least once a day, at the same hour. The health condition of the animals was recorded.

### Results

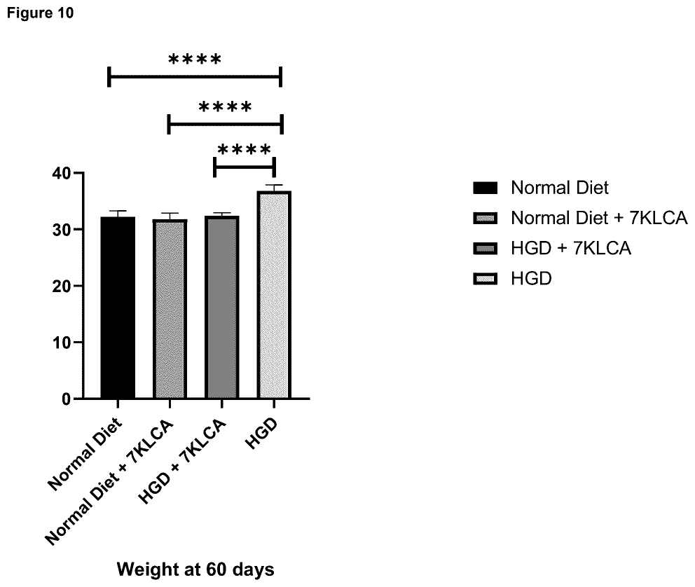

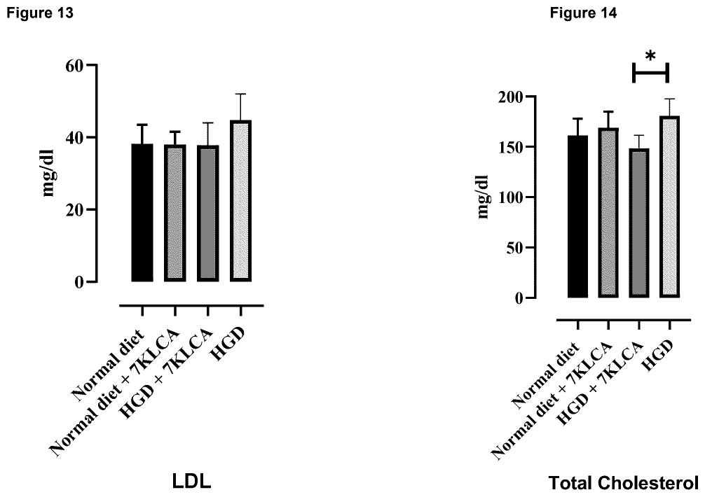

After overnight fasting, blood of the animals was collected in serum separator tubes. FIGS. 10 and 13 show the change in body weight for the mice in the study. It can be seen from FIG. 10 that, in the group of mice fed a normal diet for 60 days, there is no significant difference in the weight gains of the 5 mice in the control group (CTRL 1) and the 5 mice treated with 7KLCA at 10 mg/kg (CTRL 2). As shown in FIG. 13, in the group of mice fed a normal diet for 30 days and then switched to an HGD diet for a further 30 days, there is no significant difference in the weight gains of the 5 mice in the CTRL 3 group and the 5 mice in the treated group for the initial 30-day period in which the mice were fed a standard diet. However, when switched to the HGD diet, the mice treated with 7-KLCA gained a similar amount of weight to the mice on the normal diet (CTRL 1 and CTRL 2) but the weight gain was significantly greater in the untreated mice (CTRL 3).

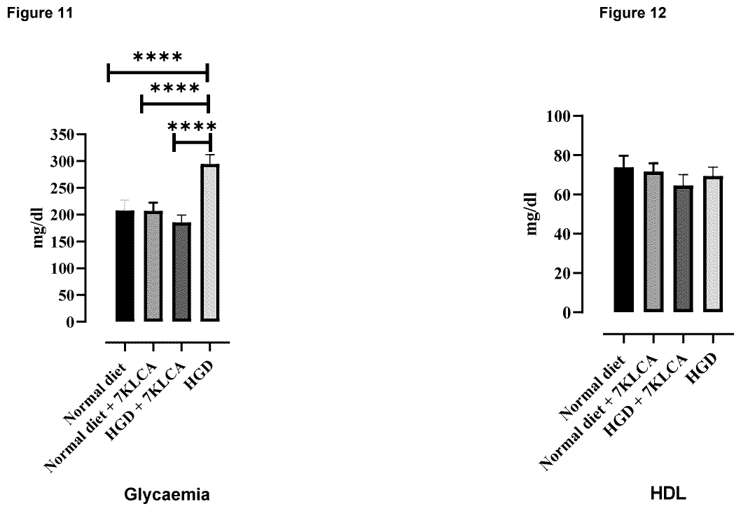

As shown in FIG. 11, in the mice fed a normal diet for 60 days, there was no significant difference in blood glucose levels between the untreated group CTRL 1 (Normal diet), and the treated control group CTRL 2 (Normal diet+7KLCA) and FIGS. 12, 13 and 14 show that there was also no significant difference in levels of HDL cholesterol, LDL cholesterol and total cholesterol between these two groups.

FIG. 11 also shows that, in the group of mice fed a normal diet for 30 days and then switched to an HGD diet for a further 30 days the blood glucose levels in the treated group (HGD+7KLCA) after 60 days were significantly lower than those in the CTRL 3 group (HGD).). As shown in FIG. 12, there was a slight, though not significant, increase in HDL cholesterol levels in the mice of the treated group (HGD+7KLCA) compared with the CTRL 3 group (HGD). The LDL cholesterol (FIG. 13) and total cholesterol (FIG. 14) levels were significantly reduced in the 7-KLCA treated mice (HGD+7KLCA) compared with the untreated mice of the CTRL3 group (HGD).

At the selected dose of 10 mg/kg, no toxicity was observed in the mice treated with 7-KLCA.

### CONCLUSIONS

The results obtained from the study showed that mice treated with 7-KLCA gained a similar amount of weight whether they were fed a normal diet (CTRL 2) or an HGD diet (Treated). The weight gain was also similar to that seen in untreated mice fed a normal diet. In contrast, untreated mice gained significantly more weight when fed an HGD diet (CTRL 3) compared with mice fed a normal diet (CTRL 1). In addition, treatment with 7-KLCA induced a statistically significant decrease in blood glucose levels with a trend of increased insulin levels in mice fed with a normal diet for 30 days followed by HGD diet for the next 30 days. The treated mice in the HGD+7KLCA group also had lower levels of LDL and total cholesterol compared with the mice of the CTRL 3 group.

### REFERENCES

Amann R. I., Binder B. J., Olson R. J., Chisholm S. W., Devereux R., Stahl D. A. (1990) “Combination of 16S ribosomal-RNA-targeted oligonucleotide probes with flow-cytometry for analyzing mixed microbial-populations”, Appl Environ Microbiol, 56, 1919-1925.
Belizário J. E. and Faintuch J. (2018) “Microbiome and Gut Dysbiosis”, Metabolic Interaction in Infection, 459-476, SpringerLink pub.
Daims H., Bruhl A., Amann R., Schleifer K. H., Wagner M. (1999) “The domain-specific probe EUB338 is insufficient for the detection of all bacteria: development and evaluation of a more comprehensive probe set”, Syst Appl Microbiol 22, 434-444.
De Vadder F., Kovatcheva-Datchary P., Goncalves D., Vinera J., Zitoun C., Duchampt A., Bäckhed F., Mithieux G. (2014) “Microbiota-generated metabolites promote metabolic benefits via gut-brain neural circuits”, Cell 156, 84-96.
Flux M., Lowry C. (2021) “Finding intestinal fortitude: Integrating the microbiome into a holistic view of depression mechanisms, treatment, and resilience”, Neurobiol. Dis. 135, 104578. Available at doi: 10.1016/j.nbd.2019.104578.
Franks A. H., Harmsen H. J., Raangs G. C., Jansen G. J., Schut F., Welling G. W. (1998) “Variations of bacterial populations in human feces measured by fluorescent in situ hybridization with group-specific 16S rRNA-targeted oligonucleotide probes”, Appl Environ Microbiol 64, 3336-3345
Gibson G. R. and Roberfroid M. B. (1995) “Dietary Modulation of the Human Colonic Microbiota: Introducing the Concept of Prebiotics”, The Journal of Nutrition, 125 (6), 1401-1412.
Gibson G., Hutkins R., Sanders M., Prescott S., Reimer R., Salminen S., Scott K., Stanton C., Swanson K., Cani P., Verbeke K. and Reid G., (2017). Expert consensus document: The International Scientific Association for Probiotics and Prebiotics (ISAPP) consensus statement on the definition and scope of prebiotics, Nature Reviews Gastroenterology & Hepatology, 14, 491-502.
Goncalves P. and Martel F. (2013) “Butyrate and colorectal cancer: the role of butyrate transport” Curr Drug Metab. 14 (9) 994-1008
Cong Guo Wei-Dong Chen, Yan-Dong Wang (2016) “TGR5, Not Only a Metabolic Regulator”, Front. Physiol. 7, Article 646.
Available at https://doi.org/10.3389/fphys.2016.00646
Guo C, Edwards J. E., Brouwer K. R., Brouwer K. L. R. (2018) “Farnesoid X Receptor Agonists Obeticholic Acid and Chenodeoxycholic Acid Increase Bile Acid Efflux in Sandwich-Cultured Human Hepatocytes: Functional Evidence and Mechanisms” J. Pharmacol. Exp. Ther., 365, 413-421. Available at https://doi.org/10.1124/jpet.117.246033
Greenblum S., Turnbaugh P. J., Borenstein E. (2012) “Metagenomic systems biology of the human gut microbiome reveals topological shifts associated with obesity and inflammatory bowel disease”, Proc. Natl. Acad. Sci. USA 109, 594-599
Gupta D., Bhatia D., Dave V., Sutariya V., Gupta S. V. (2018) “Salts of Therapeutic Agents: Chemical, Physicochemical, and Biological Considerations”, Molecules, 23, 1719-1734.
Harmsen H. J. M., Elfferich P., Schut F., Welling G. W. (1999) “A 16S rRNA-targeted probe for detection of lactobacilli and enterococci in faecal samples by fluorescent in situ hybridization”, Microb Ecol Health Dis 11, 3-12.
Harmsen H. J. M., Wildeboer-Veloo A. C. M., Grijpstra J., Knol J., Degener J. E., Welling G. W. (2000) “Development of 16S rRNA-based probes for the Coriobacterium group and the Atopobium cluster and their application for enumeration of Coriobacteriaceae in human feces from volunteers of different age groups”, Appl Environ Microbiol 66, 4523-4527.
Hill C., Guarner F., Reid G., Gibson G., Merenstein D., Pot B., Morelli L., Canani R., Flint H., Salminen S., Calder P. and Sanders M. (2014) The International Scientific Association for Probiotics and Prebiotics consensus statement on the scope and appropriate use of the term probiotic, Nature Reviews Gastroenterology & Hepatology, 11 (8), 506-514.
Kim B.-T., Kim K.-M, Kim K.-N. (2020) “The Effect of Ursodeoxycholic Acid on Small Intestinal Bacterial Overgrowth in Patients with Functional Dyspepsia: A Pilot Randomized Controlled Trial”, Nutrients, 12 (5), 1410-1420.
Kraeuter A-K., Phillips R., Samyai Z. (2020), “The Gut Microbiome in Psychosis From Mice to Men: A Systematic Review of Preclinical and Clinical Studies”, Frontiers in Psychiatry, 11, Article 799.
Available at https://doi.org/10.3389/fpsyt.2020.00799.
Langendijk P. S., Schut F., Jansen G. J., Raangs G. C., Kamphuis G. R., Wilkinson M. H. F., Welling G. W. (1995) “Quantitative fluorescence in situ hybridization of Bifidobacterium spp. with genus-specific 16S rRNA-targeted probes and its application in fecal samples), Appl Environ Microbiol 61, 3069-3075
Ma X., Fan P. X., Li L. S., Qiao S. Y, Zhang G. L., Li D. F. (2012) “Butyrate promotes the recovering of intestinal wound healing through its positive effect on the tight junctions” J. Anim. Sci. 90 (4), 266-8.
Mahmud R., Akter S., Tamanna S. K., Mazumder L., Esti I. Z., Banerjee S. Akter S., Hasan R., Acharjee M., Hossain S., Pirttia A. M. (2022), “Impact of gut microbiome on skin health: gut-skin axis observed through the lenses of therapeutics and skin diseases”, Gut Microbes, 14 (1), e2096995-1-e2096995-19.
Available at https://doi.org/10.1080/19490976.2022.2096995.
Maioli T. U., Borras-Nogues E., Torres L., Barbosa S. C., Martins V. D., Langella P., Azevedo V. A., Chatel J.-M. (2021) “Possible Benefits of Faecalibacterium prausnitzii for Obesity-Associated Gut Disorders”, Frontiers in Pharmacology, 12, Article 740636.
Manz W., Amann R., Ludwig W., Vancanneyt M., Schleifer K. H. (1996) “Application of a suite of 16S rRNA-specific oligonucleotide probes designed to investigate bacteria of the phylum cytophaga-flavobacter-bacteroides in the natural environment”, Microbiology 142, 1097-1106.
Marchesi J. R., Adams D. H., Fava F., Hermes G. D. A., Hirschfield G. M., Hold G., Quraishi M. N., Kinross J., Smidt H., Tuohy K. M., Thomas L. V., Zoetendal E. G., Hart A. (2016) “The gut microbiota and host health: a new clinical frontier”, Gut 65, 330-339.
Martinez J. E., Kahana D. D., Ghuman S., Wilson H. P., Wilson J., Kim S. c. J., Lagishetty V., Jacobs J. P., Sinha-Hikim A. P., Friedman T. C. (2021) “Unhealthy Lifestyle and Gut Dysbiosis: A Better Understanding of the Effects of Poor Diet and Nicotine on the Intestinal Microbiome” Frontiers in Endocrinology, 12, Article 667066, (2021).
Available at https://www.frontiersin.org/articles/10.3389/fendo.2021.667066
Matter K., Aijaz S., Tsapara A., Balda M. S. (2005) “Mammalian tight junctions in the regulation of epithelial differentiation and proliferation” Curr. Opin. Cell. Biol., 17 (5), 453-8
Olano-Martin E., Mountzouris K., Gibson G. and Rastall R. (2000) “In vitro fermentability of dextran, oligodextran and maltodextrin by human gut bacteria” British Journal of Nutrition, 83 (3), 247-255.
Pearson T., Caporaso J. G., Yellowhair M., Bokulich N. A., Padi M., Roe D. J., Wertheim B. C., Linhart M., Martinez J. A., Bilagody C., Hornstra H., Alberts D. S., Lance P., Thompson P. A. (2019) “Effects of ursodeoxycholic acid on the gut microbiome and colorectal adenoma development”, Cancer Medicine, 8, 617-628
Pferschy-Wenzig E.-M., Pausan M. R., Ardjomand-Woelkart K., Rock S., Ammar R. M., Kelber O., Moissl-Eichinger C., Bauer R. (2022) “Medicinal Plants and Their Impact on the Gut Microbiome in Mental Health: A Systematic Review” Nutrients 14, 2111.
Available at https://doi.org/10.3390/nu14102111.
Rajilić-Stojanović M., de Vos W. M. (2014) “The first 1000 cultured species of the human gastrointestinal microbiota” FEMS Microbiol Rev 38, 996-1047.
Ramsing N. B., Fossing H., Ferdelman T. G., Andersen F., Thamdrup B. (1996) “Distribution of bacterial populations in a stratified fjord (Mariager fjord, Denmark) quantified by in situ hybridization and related to chemical gradients in the water column”, Appl Environ Microbiol 62, 1391-1404.
Richardson, A., Calder, A., Stewart, C. and Smith, A., (1989) “Simultaneous determination of volatile and non-volatile acidic fermentation products of anaerobes by capillary gas chromatography” Letters in Applied Microbiology, 9 (1), 5-8.
Rigottier-Gois L., Bourhis A., Gramet G., Rochet V., Doré J. (2003) “Fluorescent hybridisation combined with flow cytometry and hybridisation of total RNA to analyse the composition of microbial communities in human faeces using 16S rRNA probes”, FEMS Microbiology Ecology, 43 (2), 237-245.
Rowland I., Gibson G. Heinken A. Scott K. Swann J, Thiele I., Tuohy K. (2018) “Gut microbiota functions: metabolism of nutrients and other food components” Eur. J. Nutr., 57, 1-24.
Steliou K., Boosalis M. S, Perrine S. P., Sangerman J., Faller D. V. (2012) “Butyrate histone deacetylase inhibitors”, Biores Open Access 1:192-198.
Suau A., Rochet V., Sghir A., Gramet G., Brewaeys S., Sutren M., Rigottier-Gois L., Doré J. (2001) “Fusobacterium prausnitzii and related species represent a dominant group within the human fecal flora”, Syst Appl Microbiol 24 139-145.
Tamanai-Shacoori Z., Smida I., Bousarhin L., Loreal O., Meuric V., Fong S. B. Monnaure-Mallet M., Jolivet-Gougeon A. (2017) “Roseburia spp.: a marker of health?”, Future Microbiol., 12, 157-170.
Ulsemer, P., Toutounian K., Kressel G., Goletz C., Schmidt J., Karsten U., Hahn A., Goletz S. (2016) “Impact of oral consumption of heat-treated bacteroides xylanisolvens DSM 23964 on the level of natural TFα-specific antibodies in human adults” Beneficial Microbes, 7 (4), 485-500. Available at: https://doi.org/10.3920/bm2015.0143.
Vyas U., Ranganathan N. (2012) “Probiotics, Prebiotics, and Synbiotics: Gut and Beyond”, Gastroenterology Research and Practice, 2012, Article ID 872716.
Walker A. W., Duncan S H., Leitch E. C. M., Child M. W., Flint H. J. (2005) “pH and peptide supply can radically alter bacterial populations and short-chain fatty acid ratios within microbial communities from the human colon” Appl Environ Microbiol”, 71, 3692-3700
Wallner G, Amann R, Beisker W. (1993) “Optimizing fluorescent in situ hybridization with rRNA-targeted oligonucleotide probes for flow cytometric identification of microorganisms” Cytometry 14, 136-143
Wilmanski, T., Rappaport N., Diener C., Gibbons S. M., Price N. D. (2021) “From taxonomy to metabolic output: what factors define gut microbiome health?” Gut Microbes 13 (1), e1907270-1-e1907270-20. Available at https://doi.org/10.1080/19490976.2021.1907270
Wong C. B., Odamaki T., Xiao J. (2019) “Beneficial effects of Bifidobacterium longum subsp. longum BB536 on human health: Modulation of gut microbiome as the principal action”, Journal of Functional Foods, 54, 506-519

All publications, including but not limited to patents and patent applications, cited in this specification are herein incorporated by reference as if each individual publication were specifically and individually indicated to be incorporated by reference herein as though fully set forth.

1-30. (canceled)

31. A method for the prevention or treatment of gut dysbiosis and diseases and conditions associated with poor gut health, or for improving the health of the gut microbiome, the method comprising administering to a subject an effective amount of 7-KLCA having the formula: 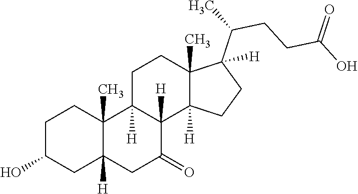
or a pharmaceutically acceptable salt, or a glycine or taurine conjugate thereof.

32. The method of claim 31, wherein the gut dysbiosis is associated with one or more diseases or conditions selected from obesity, hyperglycemia, insulin resistance, metabolic syndrome, pre-diabetes, type 2 diabetes, inflammatory bowel diseases including ulcerative colitis and Crohn's disease, irritable bowel syndrome and autoimmune conditions including rheumatoid arthritis, multiple sclerosis, type I diabetes, coeliac disease, systemic lupus erythematosus, CNS disorders including anxiety, depression, sleep disorders cognitive dysfunction, schizophrenia and bipolar disorder, cardiovascular disease and skin conditions including psoriasis, atopic dermatitis, acne vulgaris, rosacea, alopecia areata and hidradenitis suppurativa.

33. The method of claim 32, wherein the gut dysbiosis arises from the treatment of a bacterial infection with an antibiotic.

34. The method of claim 33, wherein the 7-KLCA is administered in combination with or after the administration of an antibiotic.

35. The method of claim 31, wherein the diseases and conditions associated with poor gut health are selected from obesity, hyperglycemia, insulin resistance, metabolic syndrome, pre-diabetes, type 2 diabetes, inflammatory bowel diseases including ulcerative colitis and Crohn's disease, irritable bowel syndrome and autoimmune conditions including rheumatoid arthritis, multiple sclerosis, type I diabetes, coeliac disease, systemic lupus erythematosus, CNS disorders including anxiety, depression, sleep disorders cognitive dysfunction, schizophrenia and bipolar disorder, cardiovascular disease and skin conditions including psoriasis, atopic dermatitis, acne vulgaris, rosacea, alopecia areata and hidradenitis suppurativa.

36. The method of claim 35, wherein the diseases and conditions associated with poor gut health are selected from obesity, hyperglycemia, metabolic syndrome, type 2 diabetes, inflammatory bowel diseases including ulcerative colitis and Crohn's disease, irritable bowel syndrome and autoimmune conditions including rheumatoid arthritis, multiple sclerosis, type I diabetes and coeliac disease.

37. A method of adjusting the composition of the gut microbiota of a subject to comprise a greater proportion of beneficial microorganisms, the method comprising administering to the subject 7-KLCA or a pharmaceutically acceptable salt, or a glycine or taurine conjugate thereof.

38. The method of claim 37, wherein the beneficial microorganisms comprise bacteria.

39. The method of claim 38, wherein the bacteria are selected from Bifidobacteria species, Faecalibacterium prausnitzii, Eubacterium rectale, Roseburia species, Lactobacillus species, Bacteroides xylanisolvens and Eubacterium eligens.

40. The method of claim 39, wherein the bacteria are selected from Bifidobacteria species, Faecalibacterium prausnitzii and Roseburia species.

41. The method of claim 37, further comprising adjusting the composition of the gut microbiota of the subject to comprise a lower proportion of pathogenic microorganisms.

42. The method of claim 41, wherein the pathogenic microorganisms comprise bacteria.

43. The method of claim 42, wherein the bacteria are selected from Atopobium, Coriobacterium, Clostridium histolyticum, Clostridium perfringens, Desulfovibrionales, Desulfuromonadales and Dialister pneumosintes.

44. The method of claim 37, further comprising increasing the number of microorganisms, in the gut microbiota.

45. The method of claim 37, wherein the 7-KLCA is administered simultaneously with or after antibiotic treatment.

46. A prebiotic composition comprising 7-KLCA or a pharmaceutically acceptable salt, or glycine or taurine conjugate thereof as the sole active agent together with a suitable carrier, wherein the prebiotic composition is adapted for oral administration.

47. The prebiotic composition of claim 46, wherein the composition does not contain maltodextrin.

48. The prebiotic composition of claim 47, which is a food or drink product comprising 7-KLCA or a pharmaceutically acceptable salt, or glycine or taurine conjugate thereof and a suitable flavouring.

49. The prebiotic composition of claim 46, further comprising an antibiotic.

50. A method of adjusting the composition of the gut microbiota of a subject to comprise a greater proportion of beneficial microorganisms, the method comprising administering to the subject the prebiotic composition of claim 46.
